SEQUENCE LISTING (17 sequences)

SEQ ID NO: 1-2 - skipped (2 entries)

SEQ ID NO: 3 - DNA, 4107 bp, Artificial Sequence
  source: /note=“Description of Artificial Sequence: Synthetic polynucleotide”
atggataaaaagtacagcatcgggctggacatcggtacaaactcagtggggtgggccgtgattacggacgagtacaaggtaccctccaaaaaatttaaagtgctgggtaacacggacagacactctataaagaaaaatcttattggagccttgctgttcgactcaggcgagacagccgaagccacaaggttgaagcggaccgccaggaggcggtataccaggagaaagaaccgcatatgctacctgcaagaaatcttcagtaacgagatggcaaaggttgacgatagctttttccatcgcctggaagaatcctttcttgttgaggaagacaagaagcacgaacggcaccccatctttggcaatattgtcgacgaagtggcatatcacgaaaagtacccgactatctaccacctcaggaagaagctggtggactctaccgataaggcggacctcagacttatttatttggcactcgcccacatgattaaatttagaggacatttcttgatcgagggcgacctgaacccggacaacagtgacgtcgataagctgttcatccaacttgtgcagacctacaatcaactgttcgaagaaaaccctataaatgcttcaggagtcgacgctaaagcaatcctgtccgcgcgcctctcaaaatctagaagacttgagaatctgattgctcagttgcccggggaaaagaaaaatggattgtttggcaacctgatcgccctcagtctcggactgaccccaaatttcaaaagtaacttcgacctggccgaagacgctaagctccagctgtccaaggacacatacgatgacgacctcgacaatctgctggcccagattggggatcagtacgccgatctctttttggcagcaaagaacctgtccgacgccatcctgttgagcgatatcttgagagtgaacaccgaaattactaaagcaccccttagcgcatctatgatcaagcggtacgacgagcatcatcaggatctgaccctgctgaaggctcttgtgaggcaacagctccccgaaaaatacaaggaaatcttctttgaccagagcaaaaacggctacgctggctatatagatggtggggccagtcaggaggaattctataaattcatcaagcccattctcgagaaaatggacggcacagaggagttgctggtcaaacttaacagggaggacctgctgcggaagcagcggacctttgacaacgggtctatcccccaccagattcatctgggcgaactgcacgcaatcctgaggaggcaggaggatttttatccttttcttaaagataaccgcgagaaaatagaaaagattcttacattcaggatcccgtactacgtgggacctctcgcccggggcaattcacggtttgcctggatgacaaggaagtcagaggagactattacaccttggaacttcgaagaagtggtggacaagggtgcatctgcccagtctttcatcgagcggatgacaaattttgacaagaacctccctaatgagaaggtgctgcccaaacattctctgctctacgagtactttaccgtctacaatgaactgactaaagtcaagtacgtcaccgagggaatgaggaagccggcattccttagtggagaacagaagaaggcgattgtagacctgttgttcaagaccaacaggaaggtgactgtgaagcaacttaaagaagactactttaagaagatcgaatgttttgacagtgtggaaatttcaggggttgaagaccgcttcaatgcgtcattggggacttaccatgatcttctcaagatcataaaggacaaagacttcctggacaacgaagaaaatgaggatattctcgaagacatcgtcctcaccctgaccctgttcgaagacagggaaatgatagaagagcgcttgaaaacctatgcccacctcttcgacgataaagttatgaagcagctgaagcgcaggagatacacaggatggggaagattgtcaaggaagctgatcaatggaattagggataaacagagtggcaagaccatactggatttcctcaaatctgatggcttcgccaataggaacttcatgcaactgattcacgatgactctcttaccttcaaggaggacattcaaaaggctcaggtgagcgggcagggagactcccttcatgaacacatcgcgaatttggcaggttcccccgctattaaaaagggcatccttcaaactgtcaaggtggtggatgaattggtcaaggtaatgggcagacataagccagaaaatattgtgatcgagatggcccgcgaaaaccagaccacacagaagggccagaaaaatagtagagagcggatgaagaggatcgaggagggcatcaaagagctgggatctcagattctcaaagaacaccccgtagaaaacacacagctgcagaacgaaaaattgtacttgtactatctgcagaacggcagagacatgtacgtcgaccaagaacttgatattaatagactgtccgactatgacgtagaccatatcgtgccccagtccttcctgaaggacgactccattgataacaaagtcttgacaagaagcgacaagaacaggggtaaaagtgataatgtgcctagcgaggaggtggtgaaaaaaatgaagaactactggcgacagctgcttaatgcaaagctcattacacaacggaagttcgataatctgacgaaagcagagagaggtggcttgtctgagttggacaaggcagggtttattaagcggcagctggtggaaactaggcagatcacaaagcacgtggcgcagattttggacagccggatgaacacaaaatacgacgaaaatgataaactgatacgagaggtcaaagttatcacgctgaaaagcaagctggtgtccgattttcggaaagacttccagttctacaaagttcgcgagattaataactaccatcatgctcacgatgcgtacctgaacgctgttgtcgggaccgccttgataaagaagtacccaaagctggaatccgagttcgtatacggggattacaaagtgtacgatgtgaggaaaatgatagccaagtccgagcaggagattggaaaggccacagctaagtacttcttttattctaacatcatgaatttttttaagacggaaattaccctggccaacggagagatcagaaagcggccccttatagagacaaatggtgaaacaggtgaaatcgtctgggataagggcagggatttcgctactgtgaggaaggtgctgagtatgccacaggtaaatatcgtgaaaaaaaccgaagtacagaccggaggattttccaaggaaagcattttgcctaaaagaaactcagacaagctcatcgcccgcaagaaagattgggaccctaagaaatacgggggatttgactcacccaccgtagcctattctgtgctggtggtagctaaggtggaaaaaggaaagtctaagaagctgaagtccgtgaaggaactcttgggaatcactatcatggaaagatcatcctttgaaaagaaccctatcgatttcctggaggctaagggttacaaggaggtcaagaaagacctcatcattaaactgccaaaatactctctcttcgagctggaaaatggcaggaagagaatgttggccagcgccggagagctgcaaaagggaaacgagcttgctctgccctccaaatatgttaattttctctatctcgcttcccactatgaaaagctgaaagggtctcccgaagataacgagcagaagcagctgttcgtcgaacagcacaagcactatctggatgaaataatcgaacaaataagcgagttcagcaaaagggttatcctggcggatgctaatttggacaaagtactgtctgcttataacaagcaccgggataagcctattagggaacaagccgagaatataattcacctctttacactcacgaatctcggagcccccgccgccttcaaatactttgatacgactatcgaccggaaacggtataccagtaccaaagaggtcctcgatgccaccctcatccaccagtcaattactggcctgtacgaaacacggatcgacctctctcaactgggcggcgactag

SEQ ID NO: 4-6 - skipped (3 entries)

SEQ ID NO: 7 - DNA, 3159 bp, Artificial Sequence
  source: /note=“Description of Artificial Sequence: Synthetic polynucleotide”
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 8 - DNA, 3159 bp, Artificial Sequence
  source: /note=“Description of Artificial Sequence: Synthetic polynucleotide”
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 9 - DNA, 3159 bp, Artificial Sequence
  source: /note=“Description of Artificial Sequence: Synthetic polynucleotide”
atgaagcgcaactacatcctcggactggacatcggcattacctccgtgggatacggcatcatcgattacgaaactagggatgtgatcgacgctggagtcaggctgttcaaagaggcgaacgtggagaacaacgaggggcggcgctcaaagaggggggcccgccggctgaagcgccgccgcagacatagaatccagcgcgtgaagaagctgctgttcgactacaaccttctgaccgaccactccgaactttccggcatcaacccatatgaggctagagtgaagggattgtcccaaaagctgtccgaggaagagttctccgccgcgttgctccacctcgccaagcgcaggggagtgcacaatgtgaacgaagtggaagaagataccggaaacgagctgtccaccaaggagcagatcagccggaactccaaggccctggaagagaaatacgtggcggaactgcaactggagcggctgaagaaagacggagaagtgcgcggctcgatcaaccgcttcaagacctcggactacgtgaaggaggccaagcagctcctgaaagtgcaaaaggcctatcaccaacttgaccagtcctttatcgatacctacatcgatctgctcgagactcggcggacttactacgagggtccaggggagggctccccatttggttggaaggatattaaggagtggtacgaaatgctgatgggacactgcacatacttccctgaggagctgcggagcgtgaaatacgcatacaacgcagacctgtacaacgcgctgaacgacctgaacaatctcgtgatcacccgggacgagaacgaaaagctcgagtattacgaaaagttccagattattgagaacgtgttcaaacagaagaagaagccgacactgaagcagattgccaaggaaatcctcgtgaacgaagaggacatcaagggctatcgagtgacctcaacgggaaagccggagttcaccaatctgaaggtctaccacgacatcaaagacattaccgcccggaaggagatcattgagaacgcggagctgttggaccagattgcgaagattctgaccatctaccaatcctccgaggatattcaggaagaactcaccaacctcaacagcgaactgacccaggaggagatagagcaaatctccaacctgaagggctacaccggaactcataacctgagcctgaaggccatcaacttgatcctggacgagctgtggcacaccaacgataaccagatcgctattttcaatcggctgaagctggtccccaagaaagtggacctctcacaacaaaaggagatccctactacccttgtggacgatttcattctgtcccccgtggtcaagagaagcttcatacagtcaatcaaagtgatcaatgccattatcaagaaatacggtctgcccaacgacattatcattgagctcgcccgcgagaagaactcgaaggacgcccagaagatgattaacgaaatgcagaagaggaaccgacagactaacgaacggatcgaagaaatcatccggaccaccgggaaggaaaacgcgaagtacctgatcgaaaagatcaagctccatgacatgcaggaaggaaagtgtctgtactcgctggaggccattccgctggaggacttgctgaacaacccttttaactacgaagtggatcatatcattccgaggagcgtgtcattcgacaattccttcaacaacaaggtcctcgtgaagcaggaggaaaactcgaagaagggaaaccgcacgccgttccagtacctgagcagcagcgactccaagatttcctacgaaaccttcaagaagcacatcctcaacctggcaaaggggaagggtcgcatctccaagaccaagaaggaatatctgctggaagaaagagacatcaacagattctccgtgcaaaaggacttcatcaaccgcaacctcgtggatactagatacgctactcggggtctgatgaacctcctgagaagctactttagagtgaacaatctggacgtgaaggtcaagtcgattaacggaggtttcacctccttcctgcggcgcaagtggaagttcaagaaggaacggaacaagggctacaagcaccacgccgaggacgccctgatcattgccaacgccgacttcatcttcaaagaatggaagaaacttgacaaggctaagaaggtcatggaaaaccagatgttcgaagaaaagcaggccgagtctatgcctgaaatcgagactgaacaggagtacaaggaaatctttattacgccacaccagatcaaacacatcaaggatttcaaggattacaagtactcacatcgcgtggacaaaaagccgaacagggaactgatcaacgacaccctctactccacccggaaggatgacaaagggaataccctcatcgtcaacaaccttaacggcctgtacgacaaggacaacgataagctgaagaagctcattaacaagtcgcccgaaaagttgctgatgtaccaccacgaccctcagacttaccagaagctcaagctgatcatggagcagtatggggacgagaaaaacccgttgtacaagtactacgaagaaactgggaattatctgactaagtactccaagaaagataacggccccgtgattaagaagattaagtactacggcaacaagctgaacgcccatctggacatcaccgatgactaccctaattcccgcaacaaggtcgtcaagctgagcctcaagccctaccggtttgatgtgtaccttgacaatggagtgtacaagttcgtgactgtgaagaaccttgacgtgatcaagaaggagaactactacgaagtcaactccaagtgctacgaggaagcaaagaagttgaagaagatctcgaaccaggccgagttcattgcctccttctataacaacgacctgattaagatcaacggcgaactgtaccgcgtcattggcgtgaacaacgatctcctgaaccgcatcgaagtgaacatgatcgacatcacttaccgggaatacctggagaatatgaacgacaagcgcccgccccggatcattaagactatcgcctcaaagacccagtcgatcaagaagtacagcaccgacatcctgggcaacctgtacgaggtcaaatcgaagaagcacccccagatcatcaagaaggga

SEQ ID NO: 10-14 - skipped (5 entries)

SEQ ID NO: 15 - DNA, 20 bp, Artificial Sequence
  source: /note=“Description of Artificial Sequence: Synthetic oligonucleotide”
taacctggaggatctgatcc

SEQ ID NO: 16 - DNA, 26 bp, Artificial Sequence
  source: /note=“Description of Artificial Sequence: Synthetic oligonucleotide”
taacctggaggatctgatccgggaga

SEQ ID NO: 17 - DNA, 26 bp, Artificial Sequence
  source: /note=“Description of Artificial Sequence: Synthetic oligonucleotide”
taacctggaggatctgatccgggagc

(19) United States
(12) Patent Application Publication
(10) Pub. No.: US 2021/0047633 A1
(43) Pub. Date: Feb. 18, 2021

(54) SELECTION METHODS

(71) Applicant: Editas Medicine, Inc., Cambridge, MA (US)

(72) Inventor: Barrett Ethan Steinberg, Somerville, MA (US)

(21) Appl. No.: 16/499,020

(22) PCT Filed: Mar. 29, 2018
(86) PCT No.: PCT/US2018/025282

§ 371 Date: Sep. 27, 2019

Related U.S. Application Data
(60) Provisional application No. 62/478,560, filed on Mar. 29, 2017.

(51) Int. Cl. C12N 15/10; C12N 9/22

(52) U.S. Cl. CPC C12N 15/1058; C12N 9/22

(57) ABSTRACT

The present disclosure relates to methods of selection of polynucleotides or polypeptides of interest.

### CROSS REFERENCE TO RELATED APPLICATIONS

This application claims benefit under 35 U.S.C. § 119(e) of U.S. Provisional Application No. 62/478,560, filed Mar. 29, 2017, the contents of which are incorporated herein by reference in its entirety.

### BACKGROUND

Currently available methods of selection and evolution of polypeptides and polynucleotides are typically low throughput, in that they involve a significant investment of time and/or resources with few desirable results in return.

### SUMMARY

The present invention encompasses the insight that current methods of selecting polypeptides and/or polynucleotides based on their ability to bind DNA could be improved. For example, many current methods of selection are time-consuming, requiring many rounds of selection before desirable mutants emerge. Furthermore, many current methods are not true selection methods in that they involve screening rather than selecting among mutants.

In one aspect, the present disclosure provides a system for selecting a version of an RNA guided nuclease, comprising: a library of polynucleotides, wherein different polynucleotides in the library encode different versions of the RNA guided nuclease; and a plurality of bacteriophage comprising a phagemid that encodes a first selection agent and includes a DNA target site, wherein the DNA target site comprises (i) a canonical or non-canonical protospacer adjacent motif (PAM) and (ii) a target site for a guide RNA, wherein the library of polynucleotides or the plurality of bacteriophage further comprises the guide RNA, and such that, upon incubation of (1) host cells transformed with the library with (2) the plurality of bacteriophage infect the transformed host cells.

In some embodiments, when the plurality of bacteriophage infect the transformed host cells, expression of the first selection agent confers a survival disadvantage in infected host cells and binding at the DNA target site decreases expression of the first selection agent, and host cells that were transformed with a plasmid that encodes a version of the RNA guided nuclease that binds the DNA target site survive while host cells that were transformed with a plasmid that encodes a version of the RNA guided nuclease that does not bind the DNA target site do not survive.

In some embodiments, when the plurality of bacteriophage infect the transformed host cells, expression of the first selection agent confers a survival advantage in infected host cells and binding at the DNA target site decreases expression of the first selection agent, and host cells that were transformed with a plasmid that encodes a version of the RNA guided nuclease that does not bind the DNA target site survive while host cells that were transformed with a plasmid that encodes a version of the RNA guided nuclease that binds the DNA target site do not survive.

In some embodiments, when the plurality of bacteriophage infect the transformed host cells, expression of the first selection agent confers a survival advantage in infected host cells and binding at the DNA target site increases expression of the first selection agent, and host cells that were transformed with a plasmid that encodes a version of the RNA guided nuclease that binds the DNA target site survive while host cells that were transformed with a plasmid that encodes a version of the RNA guided nuclease that does not bind the DNA target site do not survive.

In some embodiments, when the plurality of bacteriophage infect the transformed host cells, expression of the first selection agent confers a survival disadvantage in infected host cells and binding at the DNA target site increases expression of the first selection agent, and host cells that were transformed with a plasmid that encodes a version of the RNA guided nuclease that does not bind the DNA target site survive while host cells that were transformed with a plasmid that encodes a version of the RNA guided nuclease that binds the DNA target site do not survive.

In some embodiments, the target site comprises a non-canonical protospacer adjacent motif (PAM). In some embodiments, survival disadvantage comprises a decrease in cell growth kinetics (e.g., a growth delay) among the host cells, and cleavage of the non-canonical target sequence at least partially rescues the decrease in cell growth kinetics (e.g., a growth delay). In some embodiments, the target site comprises a sequence that is complementary to the gRNA sequence.

In one aspect, the present disclosure provides methods of selecting for a version of a polypeptide or polynucleotide of interest based on whether it binds a DNA target site, which comprise steps of (a) introducing a library of polynucleotides into host cells, wherein different polynucleotides in the library encode different versions of the polypeptide of interest or serve as templates for different versions of the polynucleotide of interest so that each transformed host cell includes a polynucleotide that encodes a version of the polypeptide of interest or serves as a template for a version of the polynucleotide of interest; (b) incubating transformed host cells from step (a) together with a plurality of bacteriophage comprising a phagemid that encodes a first selection agent and includes a DNA target site, under culture conditions such that the plurality of bacteriophage infect the transformed host cells, wherein expression of the first selection agent confers a survival advantage or disadvantage in infected host cells; and (d) selecting for host cells that survive step (b).

In some embodiments, in step (b), expression of the first selection agent confers a survival disadvantage in infected host cells and binding at the DNA target site decreases expression of the first selection agent, and host cells that were transformed with a plasmid that encodes a version of the polypeptide or polynucleotide of interest that binds the DNA target site survive while host cells that were transformed with a plasmid that encodes a version of the polypeptide or polynucleotide of interest that does not bind the DNA target site do not survive.

In some embodiments, in step (b), expression of the first selection agent confers a survival advantage in infected host cells and binding at the DNA target site decreases expression of the first selection agent, and host cells that were transformed with a plasmid that encodes a version of the polypeptide or polynucleotide of interest that does not bind the DNA target site survive while host cells that were transformed with a plasmid that encodes a version of the polypeptide or polynucleotide of interest that binds the DNA target site do not survive.

In some embodiments, in step (b), expression of the first selection agent confers a survival advantage in infected host cells and binding at the DNA target site increases expression of the first selection agent, and host cells that were transformed with a plasmid that encodes a version of the polypeptide or polynucleotide of interest that binds the DNA target site survive while host cells that were transformed with a plasmid that encodes a version of the polypeptide or polynucleotide of interest that does not bind the DNA target site do not survive.

In some embodiments, in step (b), expression of the first selection agent confers a survival disadvantage in infected host cells and binding at the DNA target site increases expression of the first selection agent, and host cells that were transformed with a plasmid that encodes a version of the polypeptide or polynucleotide of interest that does not bind the DNA target site survive while host cells that were transformed with a plasmid that encodes a version of the polypeptide or polynucleotide of interest that binds the DNA target site do not survive.

In some embodiments, the first selection agent is a polypeptide. In some embodiments, the first selection agent is a polynucleotide.

In some embodiments, binding at the DNA target site decreases expression of the first selection agent by cleaving the phagemid at or near the DNA target site.

In some embodiments, the phagemid includes a regulatory element that drives expression of the first selection agent. In some embodiments, the DNA target site is located within the regulatory element.

In some embodiments, binding at the DNA target site decreases expression of the first selection agent by repressing expression of the first selection agent.

In some embodiments, the regulatory element is an inducible regulatory element.

In some embodiments, step (b) comprises continuous incubation over a period of at least 4 hours.

In some embodiments, step (b) comprises continuous incubation over a period of at least 8 hours.

In some embodiments, step (b) comprises continuous incubation over a period of at least 12 hours.

In some embodiments, step (b) comprises continuous incubation over a period of at least 16 hours.

In some embodiments, the culture conditions include culturing transformed host cells from step (a) together with the plurality of bacteriophage in a competitive culture. In some embodiments, the competitive culture is a shaking liquid culture.

In some embodiments, the polypeptide or polynucleotide of interest is a polypeptide of interest.

In some embodiments, the polypeptide or polynucleotide of interest is a polynucleotide of interest.

In some embodiments, the library of polynucleotides is a library of plasmids.

In some embodiments, expression of the first selection agent confers a survival disadvantage.

In some embodiments, the first selection agent is a toxin. In some embodiments, the first selection agent is selected from the group consisting of ccdB, FlmA, fst, HicA, Hok, Ibs, Kid, LdrD, MazF, ParE, SymE, Tisb, TxpA/BrnT, XCV2162, yafO, Zeta and tse2. In some embodiments, the first selection agent is ccdB. In some embodiments, the first selection agent is tse2.

In some embodiments, polynucleotides in the library encode an antibiotic resistance gene, the first selection agent inhibits a product of the antibiotic resistance gene, and the culture conditions include exposure to an antibiotic to which the antibiotic resistance gene product provides resistance. In some embodiments, the antibiotic resistance gene encodes beta lactamase, the antibiotic is ampicillin or penicillin, and the first selection agent is beta lactamase inhibitory protein (BLIP).

In some embodiments, the first selection agent is encoded by an antibiotic resistance gene and the culture conditions include exposure to an antibiotic to which the antibiotic resistance gene provides resistance. In some embodiments, the antibiotic is selected from the group consisting of ampicillin, bleomycin, carbenicillin, chloramphenicol, erythromycin, kanamycin, penicillin, polymyxin B, spectinomycin, streptomycin, and tetracycline. In some embodiments, the antibiotic resistance gene encodes beta lactamase and the antibiotic is ampicillin or penicillin.

In some embodiments, the host cells are bacterial cells. In some embodiments, the host cells are E. coli cells.

In some embodiments, the bacteriophage are filamentous bacteriophage.

In some embodiments, the bacteriophage are M13 bacteriophage or a derivative thereof. In some embodiments, the bacteriophage are M13KO7 bacteriophage.

In some embodiments, the polypeptide is, or the polynucleotide of interest encodes, a nuclease. In some embodiments, the polynucleotide includes one or more regulatory elements that regulate or control expression of the polypeptide. In some embodiments, the regulatory element is an inducible regulatory element. In some embodiments, the inducible regulatory element is an inducible promoter, e.g., arabinose promoter, tac promoter, rhaBAD promoter.

In some embodiments, the nuclease is a CRISPR-associated (Cas) nuclease and polynucleotides in the library further comprise a DNA sequence that encodes a gRNA that localizes the Cas nuclease to the DNA target site. In some embodiments, the Cas nuclease is a Cas9 nuclease. In some embodiments, the Cas nuclease is a Cpf1 nuclease.

In some embodiments, the nuclease is a meganuclease.

In some embodiments, the nuclease comprises a DNA binding domain fused to a heterologous DNA cleavage domain. In some embodiments, the DNA binding domain comprises a TALE DNA binding domain, a zinc finger DNA binding domain, a meganuclease DNA binding domain, or an enzymatically inactive Cas protein. In some embodiments, the heterologous DNA cleavage domain is from a restriction endonuclease. In some embodiments, the heterologous DNA cleavage domain is a FokI nuclease domain.

In some embodiments, the nuclease is a zinc finger nuclease, TALEN, or mega-TALE.

In some embodiments, the nuclease comprises an enzymatically inactive Cas protein fused to a heterologous DNA cleavage domain.

In some embodiments, the polypeptide or polynucleotide of interest is a transcription regulator.

In some embodiments, the polypeptide or polynucleotide of interest is a transcriptional repressor.

In some embodiments, the polypeptide or polynucleotide of interest is a transcriptional activator.

In some embodiments, the polypeptide or polynucleotide of interest comprises a DNA binding domain fused to a heterologous transcriptional regulation domain.

In some embodiments, the DNA binding domain is a catalytically inactive Cas9 domain.

In some embodiments, the transcriptional regulation domain is a transcriptional activation domain.

In some embodiments, the transcriptional activation domain is VP16 or VP64.

In some embodiments, the transcriptional regulation domain is a transcriptional repression domain.

In some embodiments, the transcriptional repression domain is KRAB A, KRAB B, or KOX.

In some embodiments, the cleaving comprises cleaving one strand of the phagemid.

In some embodiments, the cleaving comprises cleaving both strands of the phagemid.

In another aspect, the present disclosure provides methods of selecting for a version of a polypeptide or polynucleotide of interest based on whether it binds to a DNA target site in the presence of a selection agent, which comprise steps of: (a) introducing a library of polynucleotides into host cells, wherein different polynucleotides in the library encode different versions of the polypeptide or polynucleotide of interest, so that each transformed host cell includes a polynucleotide that encodes a version of the polypeptide or polynucleotide of interest, wherein the host cell genome includes a DNA target site; (b) incubating transformed host cells from step (a) together with a plurality of bacteriophage comprising a phagemid that encodes a first selection agent, wherein the first selection agent is a first selection polynucleotide, under culture conditions such that the plurality of bacteriophage infect the transformed host cells, wherein binding of the DNA target site in the presence of the first selection agent confers a survival advantage or disadvantage in infected host cells; and (c) selecting for host cells that survive step (b).

In some embodiments, in step (b), binding of the DNA target site in the presence of the first selection agent confers a survival disadvantage in infected host cells, and host cells that were transformed with a plasmid that encodes a version of the polypeptide or polynucleotide of interest that does not bind the DNA target site in the presence of the first selection polynucleotide survive while host cells that were transformed with a plasmid that encodes a version of the polypeptide or polynucleotide of interest that does bind the DNA target site do not survive.

In some embodiments, in step (b), binding of the DNA target site in the presence of the first selection agent confers a survival advantage in infected host cells, and host cells that were transformed with a plasmid that encodes a version of the polypeptide or polynucleotide of interest that binds the DNA target site survive while host cells that were transformed with a plasmid that encodes a version of the polypeptide or polynucleotide of interest that does not bind the DNA target site do not survive.

In some embodiments, the first selection polynucleotide is a guide RNA for a CRISPR-associated (Cas) nuclease.

In another aspect, the present disclosure provides methods of selecting a version of a polypeptide or polynucleotide of interest based on whether it binds a DNA target site, which comprise steps of: (a) introducing a library of polynucleotides into host cells, wherein different polynucleotides in the library encode different versions of the polypeptide of interest or serve as templates for different versions of the polynucleotide of interest, so that each transformed host cell includes a polynucleotide that encodes a version of the polypeptide of interest or serves as a template for a version of the polynucleotide of interest; (b) incubating transformed host cells from step (a) together with a plurality of bacteriophage comprising a phagemid that encodes a first selection agent and includes a DNA target site, under culture conditions such that the plurality of bacteriophase infect the transformed host cells and confer an increase or a decrease in cell growth kinetics in infected host cells: and (c) selecting for host cells of step (b) that exhibit an increase in growth kinetics.

In some embodiments, in step (b), expression of the first selection agent confers a decrease in cell growth kinetics in infected host cells and binding at the DNA target site decreases expression of the first selection agent, and host cells that were transformed with a plasmid that encodes a version of the polypeptide or polynucleotide of interest that binds the DNA target site exhibit an increase in growth kinetics while host cells that were transformed with a plasmid that encodes a version of the polypeptide or polynucleotide of interest that does not bind the DNA target site exhibit a decrease in cell growth kinetics.

In some embodiments, in step (b), expression of the first selection agent confers an increase in cell growth kinetics in infected host cells and binding at the DNA target site decreases expression of the first selection agent, and host cells that were transformed with a plasmid that encodes a version of the polypeptide or polynucleotide of interest that does not bind the DNA target site exhibit an increase in growth kinetics while host cells that were transformed with a plasmid that encodes a version of the polypeptide or polynucleotide of interest that binds the DNA target site exhibit a decrease in cell growth kinetics.

In some embodiments, in step (b) expression of the first selection agent confers an increase in cell growth kinetics in infected host cells and binding at the DNA target site increases expression of the first selection agent, and host cells that were transformed with a plasmid that encodes a version of the polypeptide or polynucleotide of interest that binds the DNA target site exhibit an increase in growth kinetics while host cells that were transformed with a plasmid that encodes a version of the polypeptide or polynucleotide of interest that does not bind the DNA target site exhibit a decrease in cell growth kinetics.

In some embodiments, in step (b) expression of the first selection agent confers a decrease in cell growth in infected host cells and binding at the DNA target site increases expression of the first selection agent, and host cells that were transformed with a plasmid that encodes a version of the polypeptide or polynucleotide of interest that does not bind the DNA target site exhibit an increase in growth kinetics while host cells that were transformed with a plasmid that encodes a version of the polypeptide or polynucleotide of interest that binds the DNA target site exhibit a decrease in cell growth kinetics.

In another aspect, the present disclosure provides a library comprising a plurality of bacteriophage comprising a phagemid that encodes a first selection agent and comprises a DNA target site for a preselected polypeptide or polynucleotide of interest.

In another aspect, the present disclosure provides a library comprising a plurality of polynucleotides, wherein different polynucleotides in the library encode different versions of a polypeptide of interest or serve as templates for different versions of a polynucleotide of interest, wherein the polynucleotides further comprise an inducible promoter.

In another aspect, the present disclosure provides a system for selecting a version of a polypeptide or polynucleotide of interest based on whether it binds a DNA target site, comprising: (a) a library of polynucleotides, wherein different polynucleotides in the library encode different versions of the polypeptide of interest or serve as templates for different versions of the polynucleotide of interest; and (b) a plurality of bacteriophage comprising a phagemid that encodes a first selection agent and includes a DNA target site, such that, upon incubation of (1) host cells transformed with the library with (2) the plurality of bacteriophage the plurality of bacteriophage infect the transformed host cells and wherein expression of the first selection agent confers a survival advantage or disadvantage in infected host cells.

In some embodiments, expression of the first selection agent confers a survival disadvantage in infected host cells and binding at the DNA target site decreases expression of the first selection agent, and host cells that were transformed with a plasmid that encodes a version of the polypeptide or polynucleotide of interest that binds the DNA target site survive while host cells that were transformed with a plasmid that encodes a version of the polypeptide or polynucleotide of interest that does not bind the DNA target site do not survive.

In some embodiments, expression of the first selection agent confers a survival advantage in infected host cells and binding at the DNA target site decreases expression of the first selection agent, and host cells that were transformed with a plasmid that encodes a version of the polypeptide or polynucleotide of interest that does not bind the DNA target site survive while host cells that were transformed with a plasmid that encodes a version of the polypeptide or polynucleotide of interest that binds the DNA target site do not survive; or

In some embodiments, wherein expression of the first selection agent confers a survival advantage in infected host cells and binding at the DNA target site increases expression of the first selection agent, and host cells that were transformed with a plasmid that encodes a version of the polypeptide or polynucleotide of interest that binds the DNA target site survive while host cells that were transformed with a plasmid that encodes a version of the polypeptide or polynucleotide of interest that does not bind the DNA target site do not survive;

In some embodiments, expression of the first selection agent confers a survival disadvantage in infected host cells and binding at the DNA target site increases expression of the first selection agent, and host cells that were transformed with a plasmid that encodes a version of the polypeptide or polynucleotide of interest that does not bind the DNA target site survive while host cells that were transformed with a plasmid that encodes a version of the polypeptide or polynucleotide of interest that binds the DNA target site do not survive.

In another aspect, the present disclosure provides a system for selecting a variant nuclease capable of recognizing a non-canonical target sequence, comprising: a plurality of first polynucleotides, each first polynucleotide comprising (a) a first promoter sequence operably coupled to a sequence encoding a variant nuclease, and (b) a sequence encoding a positive selection agent; a second polynucleotide comprising (c) a second promoter sequence operably coupled to a sequence encoding a negative selection agent, and (d) the non-canonical target sequence, wherein cleavage of the second polynucleotide within or proximate to the non-canonical target sequence disrupts the coupling of the second promoter sequence and the negative selectable marker; and a plurality of cells, each comprising the second polynucleotide and at least one of the plurality of first polynucleotides.

In some embodiments, the nuclease is an RNA guided nuclease and one of the first and second polynucleotide further comprises a guide RNA. In some embodiments, the non-canonical target sequence is a non-canonical protospacer adjacent motif (PAM). In some embodiments, the second promoter sequence is an inducible promoter sequence, induction of the second promoter sequence results in a decrease in cell growth kinetics (e.g., a growth delay) among the plurality of cells, and cleavage of the non-canonical target sequence at least partially rescues the decrease in cell growth kinetics (e.g., a growth delay).

In one aspect, the disclosure provides a method of selecting a version of a polypeptide or polynucleotide of interest based on whether it binds a DNA target site, the method comprising steps of: (a) introducing a polynucleotide into a host cell, wherein the polynucleotide encodes a first selection agent and includes a DNA target site, so that each transformed host cell includes a polynucleotide that encodes a first selection agent and includes a DNA target site or serves as a template for the first selection agent and includes the DNA target site; (b) incubating transformed host cells from step (a) together with bacteriophage comprising a library of phagemid that encode different versions of a polypeptide of interest or serve as templates for different versions of the polynucleotide of interest, under culture conditions such that the plurality of bacteriophage infect the transformed host cells so that each transformed and infectected host cell includes a polynucleotide that encodes a version of the polypeptide of interest or serves as a template for a version of the polynucleotide of interest.

In some embodiments, in step (b), expression of the first selection agent confers a survival disadvantage in infected host cells and binding at the DNA target site decreases expression of the first selection agent, and host cells that were infected with a phagemid that encodes a version of the polypeptide or polynucleotide of interest that binds the DNA target site survive while host cells that were transformed with a phagemid that encodes a version of the polypeptide or polynucleotide of interest that does not bind the DNA target site do not survive.

In some embodiments, in step (b), expression of the first selection agent confers a survival advantage in infected host cells and binding at the DNA target site decreases expression of the first selection agent, and host cells that were transformed with a phagemid that encodes a version of the polypeptide or polynucleotide of interest that does not bind the DNA target site survive while host cells that were transformed with a phagemid that encodes a version of the polypeptide or polynucleotide of interest that binds the DNA target site do not survive.

In some embodiments, in step (b), expression of the first selection agent confers a survival advantage in infected host cells and binding at the DNA target site increases expression of the first selection agent, and host cells that were transformed with a phagemid that encodes a version of the polypeptide or polynucleotide of interest that binds the DNA target site survive while host cells that were transformed with a phagemid that encodes a version of the polypeptide or polynucleotide of interest that does not bind the DNA target site do not survive.

In some embodiments, in step (b), expression of the first selection agent confers a survival disadvantage in infected host cells and binding at the DNA target site increases expression of the first selection agent, and host cells that were transformed with a phagemid that encodes a version of the polypeptide or polynucleotide of interest that does not bind the DNA target site survive while host cells that were transformed with a phagemid that encodes a version of the polypeptide or polynucleotide of interest that binds the DNA target site do not survive; and

In some embodiments, the method further comprises step (c), selecting for host cells that survive step (b).

### BRIEF DESCRIPTION OF THE DRAWING

FIG. 1 depicts the results of an in vitro lysate cleavage assay of a highly selected Cas9 mutant obtained as described in Example 1. The mutant demonstrates cutting only on the CORD6 target, while the wildtype enzyme cleaves both targets (i.e., CORD6 target and wildtype target) efficiently. Bands corresponding to cleavage products are indicated by triangles.

FIG. 2 shows a schematic outlining an evolutionary strategy for selecting against nucleases that show activity at known off-target sites. In each cycle, library generation by a mutagenesis method is followed by a round of positive selection for on-target cleavage, which is followed by a round of negative selection against pooled bacteriophage containing various off-target sites.

FIG. 3 depicts an exemplary Cas9 library plasmid and a pSelect target phagemid.

FIG. 4 depicts exemplary results of positive selection using targeting and nontargeting Cas9 with tse2 as a positive selection agent.

FIG. 5 depicts exemplary results of positive selection using wild-type Cas9 and a library of mutant Cas9 with tse2 as a positive selection agent.

FIG. 6 depicts exemplary results of negative selection using wild-type Cas9 and a library of mutant Cas9 with chloramphenicol (“Cm”) as a negative selection agent.

FIG. 7 depicts exemplary results of successive rounds of positive and negative selection of a wild-type Cas to evolve a selective Cas9 mutant.

FIG. 8 shows a schematic outlining an evolutionary strategy for selecting against nucleases that show activity at known off-target sites. Phagemid libraries of Cas9 mutants were generated by mutagenesis followed by a round of positive selection for on-target cleavage, which is followed by a round of negative selection for off-target cleavage.

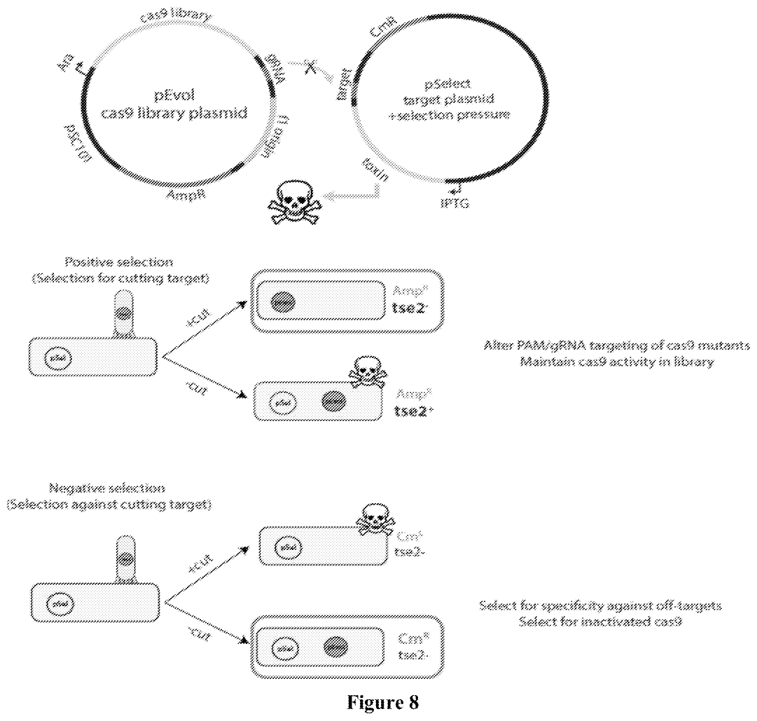

### DEFINITIONS

Throughout the specification, several terms are employed that are defined in the following paragraphs. Other definitions are also found within the body of the specification.

As used herein, the terms “about” and “approximately,” in reference to a number, is used herein to include numbers that fall within a range of 20%, 10%, 5%, or 1% in either direction (greater than or less than) of the number unless otherwise stated or otherwise evident from the context (except where such number would exceed 100% of a possible value).

As used herein, the term “cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or cohesive ends.

As used herein, a “competitive culture” refers to a growth culture in which a population of organisms (e.g., host cells) is grown together and must compete for the same limited resources, for example, nutrients, oxygen, etc.

As used herein, a “conservative substitution” refers to a substitution of an amino acid made among amino acids within the following groups: i) methionine, isoleucine, leucine, valine, ii) phenylalanine, tyrosine, tryptophan, iii) lysine, arginine, histidine, iv) alanine, glycine, v) serine, threonine, vi) glutamine, asparagine and vii) glutamic acid, aspartic acid. In some embodiments, a conservative amino acid substitution refers to an amino acid substitution that does not alter the relative charge or size characteristics of the protein in which the amino acid substitution was made.

As used herein, a “fusion protein” refers to a protein created through the joining of two or more originally separate proteins, or portions thereof. In some embodiments, a linker or spacer will be present between each protein.

As used herein, the term “heterologous,” in reference to polypeptide domains, refers to the fact that the polypeptide domains do not naturally occur together (e.g., in the same polypeptide). For example, in fusion proteins generated by the hand of man, a polypeptide domain from one polypeptide may be fused to a polypeptide domain from a different polypeptide. The two polypeptide domains would be considered “heterologous” with respect to each other, as they do not naturally occur together.

As used herein, the term “host cell” is a cell that is manipulated according to the present invention, e.g., into which nucleic acids are introduced. A “transformed host cell” is a cell that has undergone transformation such that it has taken up exogenous material such as exogenous genetic material, e.g., exogenous nucleic acids.

As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. The comparison of sequences and determination of percent identity between two sequences can be accomplished using a mathematical algorithm. As is well known in the art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999.

The term “library”, as used herein in the context of polynucleotides, refers to a population of two or more different polynucleotides. In some embodiments, a library comprises at least two polynucleotides comprising different sequences encoding nucleases and/or at least two polynucleotides comprising different sequences encoding guide RNAs. In some embodiments, a library comprises at least 10¹, at least 10², at least 10³, at least 10⁴, at least 10⁵, at least 10⁶, at least 10⁷, at least 10⁸, at least 10⁹, at least 10¹⁰, at least 10¹¹, at least 10¹², at least 10¹³, at least 10¹⁴, or at least 10¹⁵ different polynucleotides. In some embodiments, the members of the library may comprise randomized sequences, for example, fully or partially randomized sequences. In some embodiments, the library comprises polynucleotides that are unrelated to each other, e.g., nucleic acids comprising fully randomized sequences. In other embodiments, at least some members of the library may be related, for example, they may be variants or derivatives of a particular sequence.

As used herein, the term “operably linked” refers to a juxtaposition wherein the components described are in a relationship permitting them to function in their intended manner. A regulatory element “operably linked” to a functional element is associated in such a way that expression and/or activity of the functional element is achieved under conditions compatible with the regulatory element. In some embodiments, “operably linked” regulatory elements are contiguous (e.g., covalently linked) with the coding elements of interest; in some embodiments, regulatory elements act in trans to or otherwise at a from the functional element of interest.

As used herein, the term “nuclease” refers to a polypeptide capable of cleaving the phosphodiester bonds between the nucleotide subunits of nucleic acids; the term “endonuclease” refers to a polypeptide capable of cleaving the phosphodiester bond within a polynucleotide chain.

As used herein, the terms “nucleic acid”, “nucleic acid molecule” or “polynucleotide” are used herein interchangeably. They refer to a polymer of deoxyribonucleotides or ribonucleotides in either single- or double-stranded form, and unless otherwise stated, encompass known analogs of natural nucleotides that can function in a similar manner as naturally occurring nucleotides. The terms encompass nucleic acid-like structures with synthetic backbones, as well as amplification products. DNAs and RNAs are both polynucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages). As used herein, the term “oligonucleotide” refers to a string of nucleotides or analogues thereof. Oligonucleotides may be obtained by a number of methods including, for example, chemical synthesis, restriction enzyme digestion or PCR. As will be appreciated by one skilled in the art, the length of an oligonucleotide (i.e., the number of nucleotides) can vary widely, often depending on the intended function or use of the oligonucleotide. Throughout the specification, whenever an oligonucleotide is represented by a sequence of letters (chosen from the four base letters: A, C, G, and T, which denote adenosine, cytidine, guanosine, and thymidine, respectively), the nucleotides are presented in the 5′ to 3′ order from the left to the right. In certain embodiments, the sequence of an oligonucleotide includes one or more degenerate residues described herein.

As used herein, the term “off-target” refers to binding, cleavage and/or editing of an unintended or unexpected region of DNA by an RNA guided nuclease. In some embodiments, a region of DNA is an off-target region when it differs from the region of DNA intended or expected to be bound, cleaved and/or edited by 1, 2, 3, 4, 5, 6 , 7 or more nucleotides.

As used herein, the term “on-target” refers to binding, cleavage and/or editing of an intended or expected region of DNA by an RNA guided nuclease.

As used herein, the term “phagemid,” also known as “phasmid” is a plasmid that contains an f1 origin of replication from a f1 phage so that it can replicate in bacteriophage. Unless otherwise denoted, a phagemid also has an origin of replication that allows it to replicate as a plasmid in, e.g., a host cell such as a bacterial cell.

As used herein, the term “polypeptide” generally has its art-recognized meaning of a polymer of amino acids. The term is also used to refer to specific functional classes of polypeptides, such as, for example, nucleases, antibodies, etc.

As used herein, the term “regulatory element” refers to a DNA sequence that controls or impacts one or more aspects of gene expression. In some embodiments, a regulatory element is or includes a promoter, an enhancer, a silencer, and/or a termination signal. In some embodiments, a regulatory element controls or impacts inducible expression.

As used herein, the term “target site” refers to a nucleic acid sequence that defines a portion of a nucleic acid to which a binding molecule will bind, provided sufficient conditions for binding exist. In some embodiments, a target site is a nucleic acid sequence to which a nuclease described herein binds and/or that is cleaved by such nuclease. In some embodiments, a target site is a nucleic acid sequence to which a guide RNA described herein binds. A target site may be single-stranded or double-stranded. In the context of nucleases that dimerize, for example, nucleases comprising a FokI DNA cleavage domain, a target site typically comprises a left-half site (bound by one monomer of the nuclease), a right-half site (bound by the second monomer of the nuclease), and a spacer sequence between the half sites in which the cut is made. In some embodiments, the left-half site and/or the right -half site is between 10-18 nucleotides long. In some embodiments, either or both half-sites are shorter or longer. In some embodiments, the left and right half sites comprise different nucleic acid sequences. In the context of zinc finger nucleases, target sites may, in some embodiments, comprise two half-sites that are each 6-18 bp long flanking a non-specified spacer region that is 4-8 bp long. In the context of TALENs, target sites may, in some embodiments, comprise two half-sites sites that are each 10-23 bp long flanking a non-specified spacer region that is 10-30 bp long. In the context of RNA-guided (e.g., RNA-programmable) nucleases, a target site typically comprises a nucleotide sequence that is complementary to a guide RNA of the RNA-programmable nuclease, and a protospacer adjacent motif (PAM) at the 3′ end or 5′ end adjacent to the guide RNA-complementary sequence. For the RNA-guided nuclease Cas9, the target site may be, in some embodiments, 16-24 base pairs plus a 3-6 base pair PAM (e.g., NNN, wherein N represents any nucleotide). Exemplary target sites for RNA-guided nucleases, such as Cas9, are known to those of skill in the art and include, without limitation, NNG, NGN, NAG, NGA, NGG, NGAG and NGCG wherein N represents any nucleotide. In addition, Cas9 nucleases from different species (e.g., S. thermophilus instead of S. pyogenes) recognizes a PAM that comprises the sequence NGGNG. Additional PAM sequences are known, including, but not limited to NNAGAAW and NAAR (see, e.g., Esvelt and Wang, Molecular Systems Biology, 9:641 (2013), the entire contents of which are incorporated herein by reference). For example, the target site of an RNA-guided nuclease, such as, e.g., Cas9, may comprise the structure [Nz]-[PAM], where each N is, independently, any nucleotide, and z is an integer between 1 and 50. In some embodiments, z is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50. In some embodiments, z is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48,49, or 50. In some embodiments, Z is 20.

As used herein, the term “variant” refers to an entity that shows significant structural identity with a reference entity (e.g., a wild-type sequence) but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity. As will be appreciated by those skilled in the art, any biological or chemical reference entity has certain characteristic structural elements. A variant, by definition, is a distinct chemical entity that shares one or more such characteristic structural elements. To give but a few examples, a polypeptide may have a characteristic sequence element comprising a plurality of amino acids having designated positions relative to one another in linear or three-dimensional space and/or contributing to a particular biological function; a nucleic acid may have a characteristic sequence element comprising a plurality of nucleotide residues having designated positions relative to on another in linear or three-dimensional space. For example, a variant polypeptide may differ from a reference polypeptide as a result of one or more differences in amino acid sequence and/or one or more differences in chemical moieties (e.g., carbohydrates, lipids, etc.) covalently attached to the polypeptide backbone. In some embodiments, a variant polypeptide shows an overall sequence identity with a reference polypeptide (e.g., a nuclease described herein) that is at least 60%, 65%, 70%, 75%, 80%, 85%, 86%, 87%, 88%, 89%, 90%, 91%, 92%, 93%, 94%, 95%, 96%, 97%, 98% or 99%. Alternatively or additionally, in some embodiments, a variant polypeptide does not share at least one characteristic sequence element with a reference polypeptide. In some embodiments, the reference polypeptide has one or more biological activities. In some embodiments, a variant polypeptide shares one or more of the biological activities of the reference polypeptide, e.g., nuclease activity. In some embodiments, a variant polypeptide lacks one or more of the biological activities of the reference polypeptide. In some embodiments, a variant polypeptide shows a reduced level of one or more biological activities (e.g., nuclease activity, e.g., off-target nuclease activity) as compared with the reference polypeptide. In some embodiments, a polypeptide of interest is considered to be a “variant” of a parent or reference polypeptide if the polypeptide of interest has an amino acid sequence that is identical to that of the parent but for a small number of sequence alterations at particular positions. Typically, fewer than 20%, 15%, 10%, 9%, 8%, 7%, 6%, 5%, 4%, 3%, 2% of the residues in the variant are substituted as compared with the parent. In some embodiments, a variant has 10, 9, 8, 7, 6, 5, 4, 3, 2, or 1 substituted residue as compared with a parent. Often, a variant has a very small number (e.g., fewer than 5, 4, 3, 2, or 1) number of substituted functional residues (i.e., residues that participate in a particular biological activity). In some embodiments, a variant has not more than 5, 4, 3, 2, or 1 additions or deletions, and often has no additions or deletions, as compared with the parent. Moreover, any additions or deletions are typically fewer than about 25, about 20, about 19, about 18, about 17, about 16, about 15, about 14, about 13, about 10, about 9, about 8, about 7, about 6, and commonly are fewer than about 5, about 4, about 3, or about 2 residues. In some embodiments, the parent or reference polypeptide is one found in nature.

### DETAILED DESCRIPTION OF CERTAIN EMBODIMENTS

The present invention encompasses the insight that current methods of selecting polypeptides and/or polynucleotides based on their ability to bind DNA could be improved. For example, many current methods of selection are time-consuming, requiring many rounds of selection before desirable mutants emerge. Furthermore, many current methods are not true selection methods in that they involve screening rather than selecting among mutants.

In certain embodiments, the present disclosure provides more efficient methods of selecting polypeptides and/or polynucleotides by continuously presenting a fitness challenge to the variants being selected among during each round of selection.

In certain embodiments, the present disclosure provides a competitive-based selection strategy that would select for the variants (e.g., among a library of variants/mutants) having the greatest fitness in a set of conditions. Rather than screening only for variants that meet a particular threshold with respect to a fitness requirement, embodiments of the presently disclosed methods allow selection of the variants having the greatest fitness with respect to a fitness requirement.

Selection methods of the present invention are useful, for example, in directed evolution strategies, e.g., strategies that involve one or more rounds of mutagenesis followed by selection. In certain embodiments, presently disclosed methods allow a higher throughput directed evolution strategy than is typically observed with current polypeptide and/or polypeptide evolution strategies.

### Selection Methods

Selection Based on Binding to a DNA Target Site in a Phagemid

In one aspect, the present disclosure provides methods of selecting for a version of a polypeptide or polynucleotide of interest based on whether it binds a DNA target site.

As discussed further herein, these methods generally comprise steps of (a) providing a library of polynucleotides, wherein different polynucleotides in the library encode different versions of the polypeptide of interest or serve as templates for different versions of the polynucleotide of interest; (b) introducing the library of polynucleotides into host cells so that each transformed host cell includes a polynucleotide that encodes a version of the polypeptide of interest or serves as a template for a version of the polynucleotide of interest; (c) providing a plurality of bacteriophage comprising a phagemid that encodes a first selection agent and includes a DNA target site; (d) incubating transformed host cells from step (b) together with the plurality of bacteriophage under culture conditions such that the plurality of bacteriophage infect the transformed host cells, wherein expression of the first selection agent confers a survival advantage or disadvantage in infected host cells; and (e) selecting for host cells that exhibit a survival advantage (e.g., a survival advantage described herein) in (d). For example, survival of step (d) is based on various schemes as outlined below.

Binding at the DNA target site decreases expression of a selection agent (as further discussed herein) encoded by or contained on the phagemid. As discussed further herein, the selection agent may confer a survival disadvantage or a survival advantage. A decrease in the expression of the selection agent can occur by transcriptional repression of the selection agent mediated by binding at the DNA target site. In some embodiments, a decrease in the expression of the selection agent occurs by cleavage of the phagemid at or near the DNA target site. For example, the polypeptide or polynucleotide of interest can both bind to and cleave DNA. Some classes of enzymes bind to a particular DNA recognition site and cleave the DNA at or near the binding site. Accordingly, the site of DNA cleavage can be the same or different than the DNA binding site. In some embodiments in which the DNA cleavage site is different than the DNA binding site, the two sites are near one another (e.g., within 20, 15, 10, or 5 base pairs). Additionally or alternatively, the two sites are not within 20, 15, 10, or 5 base pairs of one another.

When cleavage is involved, it may be cleavage of one strand (also referred to as “nicking”) or both strands of the phagemid, which, as discussed below, replicates as double-stranded plasmid when inside host cells.

In certain embodiments, binding at the DNA target site increases expression of a selection agent encoded by the phagemid or whose template is on the phagemid. An increase in the expression of the selection agent can occur, e.g., by transcriptional activation of the selection agent mediated by binding at the DNA target site.

Versions of polypeptides or polynucleotides that bind to the DNA target site can be selected, e.g., in that host cells that were transformed with such versions exhibit a maintenance of cell growth kinetics, an increase in cell growth kinetics (e.g., an increase in cell division), and/or reversal of a decrease in cell growth kinetics (e.g., at least a partial rescue from a decrease in cell growth kinetics; while host cells that were transformed with versions that do not bind the DNA target site do not exhibit an increase in cell growth kinetics (e.g., exhibit a decrease in cell growth kinetics and/or cell division) and/or are killed. In certain embodiments, versions of polypeptides or polynucleotides that bind to the DNA target site are selected in that host cells that were transformed with such versions survive, while host cells that were transformed with versions that do not bind the DNA target site do not survive.

Versions of polypeptides or polynucleotides that do not bind to the DNA target site can be selected, e.g., in that host cells that were transformed with such versions exhibit a maintenance of cell growth kinetics, an increase in cell growth kinetics (e.g., an increase in cell division), and/or reversal of a decrease in cell growth kinetics (e.g., at least a partial rescue from a decrease in cell growth kinetics; while host cells that were transformed with versions that bind the DNA target site do not exhibit an increase in cell growth kinetics (e.g., exhibit a decrease in cell growth kinetics and/or cell division) and/or are killed. In certain embodiments, versions of polypeptides or polynucleotides that do not bind to the DNA target site are selected in that host cells that were transformed with such versions survive, while host cells that were transformed with versions that bind the DNA target site do not survive.

In an alternative embodiment, a phagemid, (e.g., pEvol_CAS), encoding a Cas9 protein and a gRNA targeting a target sequence along with a phage origin F1 element can be constructed (FIG. 17). In some embodiments, the phagemid constitutively expresses beta-lactamase, which confer resistance to ampicillin (AmpR), or a similar antibiotic such as carbecillin, and an inducible arabinose promoter (Ara) to control expression of Cas9. In some embodiments, a pEvol_CAS can be packaged into helper bacteriophage for introduction into transformed host cells. Plasmids, for example pSelect_MUT and pSelect_WT can also be constructed, each containing a potential target site. Alternatively, or additionally these plasmids may also contain a constitutively expressed chloramphenicol resistance gene (CmR) and a bacterial toxin under the control of lac promoter, allowing induction of toxin expression by, for example, IPTG (Isopropyl β-D-1-thiogalactopyranoside).

To engineer allele specificity, phagemid libraries of Cas9 mutants can be generated using, for example, a pEvol_CAS phagemid as the initial template for mutagenesis, and a comprehensive and unbiased mutagenesis method that targets every codon and allows tuning of the mutation rate.

Alternatively or additionally, in some embodiments, each round of evolution comprises subjecting a phagemid library of pEvol_CAS mutants to positive selection for cutting against E. coli containing, for example, pSelect_MUT or pSelect_WT in a competitive culture. For example, bacteria containing pSelect_MUT or pSelect_WT can be infected using phage packaging pEvol_CAS mutants and the bacteria can be cultured in ampicillin in a liquid culture.

In some embodiments, after an initial incubation and infection, the stringency of positive selection using a toxin can be assessed by adding, for example, IPTG, to induce toxin expression. In some embodiments, expression of Cas9 and guide RNA can be induced by addition of arabinose. During positive selection, bacteria can be continuously infected by phage present in the liquid culture, thus presenting a continuous challenge to cut the target.

In some embodiments, after an intial incubation and infection, the stringency of negative selection using an antibiotic, e.g., chloramphenicol. In some embodiments, expression of Cas9 and guide RNA can be induced by addition arabinose. During negative selection, bacteria can be continuously infected by phage present in the liquid culture, thus presenting the continuous challenge to not cut the target.

In another aspect, these methods generally comprise steps of (a) providing a polynucleotide that encodes a first selection agent and includes a DNA target site; (b) introducing the polynucleotides into host cells so that each transformed host cell includes a polynucleotide that encodes the first selection agent and the DNA target site; (c) providing a plurality of bacteriophage comprising phagemid that encodes a library of polynucleotides, wherein different polynucleotides in the library encode different versions of the polypeptide of interest or serve as templates for different versions of the polynucleotide of interest; (d) incubating transformed host cells from step (b) together with the plurality of bacteriophage under culture conditions such that the plurality of bacteriophage infect the transformed host cells, wherein expression of the first selection agent confers a survival advantage or disadvantage in infected host cells; and (e) selecting for host cells that exhibit a survival advantage (e.g., a survival advantage described herein) in (d). For example, survival of step (d) is based on various schemes as outlined above.

### Selection Based on Binding at the DNA Target Site When Binding Decreases Expression of a Disadvantageous Selection Agent

In certain embodiments, the selection agent confers a survival disadvantage (e.g., a decrease in cell growth kinetics (e.g., a growth delay) and/or an inhibition of cell division) in host cells and/or kills the host cells, and binding at the DNA target site decreases expression of the selection agent. Survival is then based on binding at the DNA target site: host cells that were transformed with a polynucleotide from the library that encodes a version of the polypeptide of interest, or that serves as a template for a version of the polynucleotide of interest, that binds to the DNA target site exhibit a maintenance of cell growth kinetics, an increase in cell growth kinetics (e.g., an increase in cell division), and/or reversal of a decrease in cell growth kinetics (e.g., at least a partial rescue from a decrease in cell growth kinetics). Meanwhile, host cells that were transformed with a polynucleotide from the library that encodes a version of the polypeptide of interest, or that serves as a template for a version of the polynucleotide of interest, that does not bind to the DNA target site exhibit a survival disadvantage (e.g., a decrease in cell growth kinetics (e.g., a growth delay) and/or an inhibition of cell division), do not survive and/or are killed).

In some embodiments, expression of the selection agent is decreased by cleaving the phagemid at or near the DNA target site, e.g., by cleaving one strand (“nicking”) or both strands of the phagemid.

Polynucleotides in the library can include, e.g., an antibiotic resistance gene, and the selection agent (encoded by the phagemid or whose template is on the phagemid) inhibits a product of the antibiotic resistance gene. Culture conditions during such selection can include, e.g., exposure to the antibiotic to which the antibiotic resistance gene provides resistance. In one example, the antibiotic resistance gene encodes beta lactamase, the antibiotic is ampicillin or penicillin or another beta-lactam antibiotic, and the selection agent is beta lactamase inhibitory protein (BLIP).

### Selection Based on Lack of Binding at the DNA Target Site When Binding Would Decrease Expression of an Advantageous Selection Agent

In certain embodiments, the selection agent confers a survival advantage (e.g., a maintenance of cell growth kinetics, an increase in cell growth kinetics (e.g., an increase in cell division), and/or reversal of a decrease in cell growth kinetics (e.g., at least a partial rescue from a decrease in cell growth kinetics), in host cells, and binding at the DNA target site decreases expression of the selection agent. Survival is then based on a lack of binding at the DNA target site: host cells that were transformed with a polynucleotide from the library that encodes a version of the polypeptide of interest, or that serves as a template for a version of the polynucleotide of interest, that does not bind to the DNA target site exhibit a maintenance of cell growth kinetics, an increase in cell growth kinetics (e.g., an increase in cell division), and/or reversal of a decrease in cell growth kinetics (e.g., at least a partial rescue from a decrease in cell growth kinetics). Meanwhile, host cells that were transformed with a polynucleotide form the library that encodes a version of the polypeptide of interest, or that serves as a template for a version of the polynucleotide of interest, that binds to the DNA target site exhibit a survival disadvantage (e.g., a decrease in cell growth kinetics (e.g., a growth delay), an inhibition of cell division, do not survive and/or are killed).

In some embodiments, expression of the selection agent is decreased by cleaving the phagemid at or near the DNA target site, e.g., by cleaving one strand (“nicking”) or both strands of the phagemid.

### Selection Based on Binding at the DNA Target Site When Binding Increases Expression of an Advantageous Selection Agent

In certain embodiments, the selection agent confers a survival advantage (e.g., a maintenance of cell growth kinetics, an increase in cell growth kinetics, (e.g., an increase in cell division), and/or reversal of a decrease in cell growth kinetics (e.g., at least a partial rescue from a decrease in cell growth kinetics) in host cells, and binding at the DNA target site increases expression of the selection agent. Survival is then based on binding at the DNA target site: host cells that were transformed with a polynucleotide from the library that encodes a version of the polypeptide of interest, or that serves as a template for a version of the polynucleotide of interest, that binds to the DNA target site exhibit a maintenance of cell growth kinetics, an increase in cell growth kinetics (e.g., an increase in cell division), and/or reversal of a decrease in cell growth kinetics (e.g., at least a partial rescue from a decrease in cell growth kinetics). Meanwhile, host cells that were transformed with a polynucleotide form the library that encodes a version of the polypeptide of interest, or that serves as a template for a version of the polynucleotide of interest, that does not bind to the DNA target site exhibit a survival disadvantage (e.g., a decrease in cell growth kinetics (e.g., a growth delay), an inhibition of cell division, do not survive and/or are killed).

### Selection Based on Lack of Binding at the DNA Target Site When Binding Would Increase Expression of an Disadvantageous Selection Agent

In certain embodiments, the selection agent confers a survival disadvantage (e.g., a decrease in cell growth kinetics (e.g., a growth delay) and/or an inhibition of cell division) in host cells and/or kills the host cells, and binding at the DNA target site increases expression of the selection agent. Survival is then based on a lack of binding at the DNA target site: host cells that were transformed with a polynucleotide from the library that encodes a version of the polypeptide of interest, or that serves as a template for a version of the polynucleotide of interest, that does not bind to the DNA target site exhibit a maintenance of cell growth kinetics, an increase in cell growth kinetics (e.g., an increase in cell division), and/or reversal of a decrease in cell growth kinetics (e.g., at least a partial rescue from a decrease in cell growth kinetics). Meanwhile, host cells that were transformed with a polynucleotide form the library that encodes a version of the polypeptide of interest, or that serves as a template for a version of the polynucleotide of interest, that binds to the DNA target site exhibit a survival disadvantage (e.g., a decrease in cell growth kinetics (e.g., a growth delay), an inhibition of cell division, do not survive and/or are killed).

Selection Based on Binding to a DNA Target Site in the Host Cell Genome in the Presence of a Selection Agent

In one aspect, the present disclosure provides methods of selecting for a version of a polypeptide or polynucleotide of interest based on whether it binds to a DNA target site in the presence of a selection agent.

As discussed further herein, these methods generally comprise steps of: (a) providing a library of polynucleotides, wherein different polynucleotides in the library encode different versions of the polypeptide or polynucleotide of interest; (b) introducing the library of polynucleotides into host cells so that each transformed host cell includes a polynucleotide that encodes a version of the polypeptide or polynucleotide of interest, wherein the host cell genome includes a DNA target site; (c) providing a plurality of bacteriophage comprising a phagemid that encodes a first selection agent, wherein the first selection agent is a first selection polynucleotide; (d) incubating transformed host cells from step (b) together with the plurality of bacteriophage under culture conditions such that the plurality of bacteriophage infect the transformed host cells, wherein binding of the DNA target site in the presence of the first selection agent confers a survival advantage or disadvantage in infected host cells; and (e) selecting for host cells that survive step (d).

Survival of step (d) is based on various schemes as outlined below.

The DNA target site can be, e.g., in the host cell genome. Additionally or alternatively, the DNA target site can be in an essential survival gene of the host cell. Additionally or alternatively, the DNA target site can be in a gene whose product prevents a survival gene from being expressed.

In some embodiments, the selection polynucleotide is a guide RNA for a CRISPR-associated (Cas) nuclease.

### Survival Based on Lack of Binding at the DNA Target Site When Binding Would be Disadvantageous

In certain embodiments, binding at the DNA target site in the host cell in the presence of the selection agent (which is a polynucleotide) is disadvantageous (e.g., because binding at the DNA target site results in disruption of an essential survival gene in the host cell). Survival is then based on a lack of binding at the DNA target site: host cells that were transformed with a polynucleotide from the library that encodes a version of the polypeptide of interest, or that serves as a template for a version of the polynucleotide of interest, that does not bind to the DNA target site survive. Meanwhile, host cells that were transformed with a polynucleotide form the library that encodes a version of the polypeptide of interest, or that serves as a template for a version of the polynucleotide of interest, that binds to the DNA target site do not survive.

### Survival Binding at the DNA Target Site When Binding Would be Advantageous

In certain embodiments, binding at the DNA target site in the host cell in the presence of the selection agent (which is a polynucleotide) is advantageous (e.g., because binding at the DNA target site results expression of a survival gene in the host cell). Survival is then based on binding at the DNA target site: host cells that were transformed with a polynucleotide from the library that encodes a version of the polypeptide of interest, or that serves as a template for a version of the polynucleotide of interest, that binds to the DNA target site survive. Meanwhile, host cells that were transformed with a polynucleotide form the library that encodes a version of the polypeptide of interest, or that serves as a template for a version of the polynucleotide of interest, that does not bind to the DNA target site do not survive.

Selection Based on Induction of Expression of a Polypeptide

In one aspect, the present disclosure provides methods of selecting for a version of a polypeptide or polynucleotide of interest based on modulating or controlling the expression of the polypeptide.

As discussed further herein, these methods generally comprise steps of (a) providing a library of polynucleotides, wherein different polynucleotides in the library encode different versions of the polypeptide of interest or serve as templates for different versions of the polynucleotide of interest; (b) introducing the library of polynucleotides into host cells so that each transformed host cell includes a polynucleotide that encodes a version of the polypeptide of interest or serves as a template for a version of the polynucleotide of interest; (c) inducing expression of the polypeptide to control the amount of polypeptide that is present in the culture; (d) providing a plurality of bacteriophage comprising a phagemid that encodes a first selection agent and includes a DNA target site; (e) incubating transformed host cells from step (b) together with the plurality of bacteriophage under culture conditions such that the plurality of bacteriophage infect the transformed host cells, wherein expression of the first selection agent confers a survival advantage or disadvantage in infected host cells; and (f) selecting for host cells that survive step (d). Survival of step (d) is based on various schemes described herein.

The polynucleotide can include an inducible promoter, e.g., an inducible promoter described herein, and expression is induced by contacting the polynucleotide with one or more induction agents described herein. For example, a polynucleotide can include an arabinose promoter, and expression from the polynucleotide can be induced by contacting the polynucleotide with arabinose. In another example, a polynucleotide can include a tac promoter, and expression from the polynucleotide can be induced by contacting the polynucleotide with IPTG. In yet another example, a polynucleotide can include a rhaBAD promoter, and expression from the polynucleotide can be induced by contacting the polynucleotide with rhamnose.

### Libraries of Polynucleotides

Methods of the present disclosure can start, e.g., with a step of providing a library of polynucleotides (such as a plasmid library), in which different polynucleotides in the library encode different versions of polypeptide of interest (or, in the case of a polynucleotide of interest, the library includes different versions of a polynucleotide of interest and/or different versions of a polynucleotide of interest that serve as a template for different versions of the polynucleotide of interest).

A library described herein can include, e.g., polynucleotides operably linked to an inducible promotor. For example, induction of a promoter can induce expression of a polypeptide encoded by a polynucleotide.

In some embodiments, induction of a promoter to induce expression of a polypeptide encoded by a polynucleotide affects efficiency of a selection method. For example, efficiency of a selection method can be improved and/or increased relative to efficiency of a selection method that does not use an inducible promoter.

Such libraries may be obtained, e.g., by using or purchasing an existing library, such as one that is commercially available and/or available through public collections.

Alternatively or additionally, the library may be obtained from a mutagenesis method.

For example, the library can be obtained by a random mutagenesis method or by a comprehensive mutagenesis method, e.g., a method that randomly targets a polynucleotide throughout an entire pre-defined target region for mutagenesis.

A library can also be obtained by a targeted mutagenesis method. For example, a subregion of the polynucleotide of interest, or of the polypeptide of interest, can be targeted for mutagenesis. Additionally or alternatively, the entire polynucleotide of interest, or the entire polypeptide of interest, can be targeted for mutagenesis.

Although polypeptides or polynucleotides of interest typically have DNA-binding ability, it is expected that not all versions of the polypeptide or polynucleotide of interest encoded by the different polynucleotides in the library would necessarily be able to bind DNA. Furthermore, among those versions of polypeptide or polynucleotide of interest encoded by the different polynucleotides in the library, it is expected that they may have differing abilities to bind DNA. Indeed, selection methods of the present disclosure involve distinguishing between versions of the polypeptide or polynucleotide of interest that can and cannot bind to a DNA target site. In certain embodiments, many or even most of the versions of the polypeptide or polynucleotide of interest do not bind to DNA.

Similarly, in embodiments in which the polypeptide or polynucleotide of interest can cleave DNA, not all of the versions of the polypeptide or polynucleotide of interest can necessarily cleave DNA.

### Host Cells

Methods of the present disclosure can comprise, after the step of providing a library of polynucleotides, introducing the library of polynucleotides into host cells, so that each transformed host cell includes a polynucleotide that encodes a version of the polypeptide of interest or serves as a template for a version of the polynucleotide of interest.

Host cells generally refer cells that can take up exogenous materials, e.g., nucleic acids (such as DNA and RNA), polypeptides, or ribonuclear proteins.

Host cells can be, e.g., single cell organisms, such as, e.g., microorganisms or eukaryotic cells, e.g., yeast cells, mammalian cells (e.g., in culture) etc.

In some embodiments, host cells are prokaryotic cells, e.g., bacterial cells, e.g., E. coli bacteria. Bacterial cells can be Gram-negative or Gram-positive and can belong to the Bacteria (formerly called Eubacteria) domain or the Archaea (formerly called Archaebacteria) domain. Any of these types of bacteria may be suitable as host cells so long as they can be grown in a laboratory setting and can take up exogenous materials.

The host cells can be bacterial cells that are competent or made competent, e.g., in that they are able or made to be able to take up exogenous material such as genetic material.

There a variety of mechanisms by which exogenous materials such as genetic material can be introduced into host cells. For example, in bacteria, there are three general mechanisms, classified as transformation (uptake and incorporation of extracellular nucleic acids such as DNA), transduction (e.g., transfer of genetic material from one cell to another by a plasmid or by a virus that infects the cells, like bacteriophage), and conjugation (direct transfer of nucleic acids between two cells that are temporarily joined). Host cells into which genetic material have been introduced by transformation are generally referred to as “transformed host cells.”

In some embodiments, the library of polynucleotides is introduced into host cells by transformation. Protocols for transforming host cells are known in the art. For bacterial cells, for example, there are methods based on electroporation, methods based in lipofection, methods based on heat shock, methods based on agitation with glass beads, methods based on chemical transformation, methods based on bombardment with particles coated with exogenous material (such as DNA or RNA, etc.). One of ordinary skill in the art will be able to choose a method based on the art and/or protocols provided by manufacturers of the host cells.

Transformed host cells, e.g., can each contain a polynucleotide that encodes a version of the polypeptide of interest or serves as a template for a version of polynucleotide of interest.

A library of polynucleotides can be introduced into a population of host cells such that the population of transformed host cells collectively contain all members of the library. That is, for every version of polynucleotide in the library, at least one host cell in the population contains that version of the polynucleotide, such that all versions of the polynucleotide in the library are represented in the population of transformed host cells.

### Bacteriophage

Methods of the present disclosure can comprise, after the step introducing the library of polynucleotides into host cells, providing a plurality of bacteriophage comprising a phagemid that encodes a first selection agent and includes a DNA target site.

Bacteriophage are viruses that infect bacteria and inject their genomes (and/or any phagemids packaged within the bacteriophage) into the cytoplasm of the bacteria. Generally, bacteriophage replicate within the bacteria, though replication-defective bacteriophage exist.

In some embodiments, a plurality of bacteriophage comprising a phagemid as described herein is incubated together with transformed host cells under conditions that allow the bacteriophage to infect the transformed host cells. The bacteriophage can be replication-competent, e.g., the bacteriophage replicate within the transformed host cells, and the replicated viral particles are released as virions in the culture medium, allowing re-infection of other host cells by bacteriophage.

Virions can be released from the host cells without lysing the host cells.

In some embodiments, the plurality of bacteriophage continuously infects (infects and re-infects) transformed host cells, thereby presenting a continuous challenge to the host cell.

Bacteriophage can be “helper phage” in that they preferentially package phagemid over phage DNA. For example, the bacteriophage can preferentially packages phagemid over phage DNA by a factor of at least 3:1, at least 4:1, at least 5:1, at least 6:1, at least 7:1, at least 8:1, at least 9:1, or at least 10:1.

In some embodiments, the bacteriophage do not generally lyse their host cells, e.g., the bacteriophage do not lyse their host cells under the conditions in which the transformed host cells are incubated together with the plurality of bacteriophage.

The bacteriophage can be filamentous bacteriophage. Filamentous bacteriophage usually infect Gram-negative bacteria (which include, among other things, E. coli, P. aeruginosa, N. gonorrhoeae, and Y. pestis) and have a genome of single-stranded DNA.

For example, the filamentous bacteriophage are Ff phage, which infect E. coli that carry the F episome. Examples of such phage include, but are not limited to, M13 bacteriophage, flphage, fd phage, and derivatives and variants thereof.

Additionally or alternatively, the bacteriophage can be an M13 bacteriophage or a derivative or variant thereof, e.g., the bacteriophage can be M13KO7, a derivative of M13 that has a kanamycin resistance marker and a p15A origin of replication. M13KO7 has been characterized has having a high phagemid versus phage packing ratio of approximately 10:1, thereby serving as a useful helper phage.

Additionally or alternatively, the bacteriophage can be VCSM13, a derivative of M13KO7.

The bacteriophage can also be an f1 bacteriophage or a derivative or variant thereof. For example, the bacteriophage can be R408, a derivative of f1 that does not have any antibiotic selection marker.

Additionally or alternatively, the bacteriophage can be CM13, a derivative of M13KO7 that has been reported to produce virions more reliably than M13KO7.

Pools of bacteriophage containing different phagemids can also be used in methods of the disclosure. For example, as discussed further herein, different off-site targets can be presented on different phagemids contained in the same pool of bacteriophage when it is desired, for example, to select against binding and/or cleaving at more than one off-target site.

### Phagemids

Phagemids are circular plasmids that have an f1 origin of replication from an f1 phage, and therefore can be replicated as a plasmid and packaged as single-stranded DNA by bacteriophage. Phagemids also contain an origin of replication for double-stranded replication (e.g., while inside a host cell).

Phagemids suitable for use in the present invention generally encode, or serve as a template for, a selection agent and comprise a DNA target site. Thus, phagemids for use in the present invention typically comprise a regulatory element operably linked to, and driving expression of, a gene element encoding, or serving as a template for, the selection agent.

As noted above, the DNA target site can be included anywhere within the phagemid. For example, the DNA target site can be located within the regulatory element, within the gene element, outside of and distal to both the regulatory element and the gene element, or outside of both elements but near at least one of the elements.

The position of the DNA target site may depend on the embodiment. For example, the DNA target site can be located within the regulatory element. This positioning may be suitable, for example, in embodiments in which the polypeptide of interest is a transcription factor, e.g., a transcriptional activator or repressor, and selection is based on whether or not the transcription factor binds to the DNA target site.

There is no restriction on where the DNA target site may be located, in that binding of the polypeptide of interest anywhere within the phagemid will increase or decrease expression of the selection agent. For example, binding of the polypeptide of interest at the DNA target site can result in cleaving of the phagemid at or near the DNA target site. Cleavage of the phagemid anywhere within the phagemid would cause linearization of the phagemid, which would result in the phagemid not being replicated within the host cell, therefore abrogating expression of the selection agent.

Phagemids can be packaged into bacteriophage using methods known in the art, including protocols provided by manufacturers of the bacteriophage. For example, a commonly used protocol is to make a double-stranded plasmid version of the desired phagemid construct, transform the double-stranded plasmid into host cells such as bacteria, and then inoculate a culture of such transformed host cells with helper bacteriophage, which may package the double-stranded plasmid as a single-stranded phagemid.

### Culture Conditions

Methods of the present disclosure can comprise, after the step of providing a plurality of bacteriophage comprising a phagemid that encodes a first selection agent and includes a DNA target site, a step of incubating transformed host cells (into which the library of polynucleotides was introduced) together with a plurality of bacteriophage under culture conditions such that the plurality of bacteriophage infect the transformed host cells. Generally, these conditions are conditions in which expression of the first selection agent confers either a survival disadvantage or a survival advantage, depending on the embodiment.

In certain embodiments, the culture conditions are competitive culture conditions.

“Competitive culture conditions” refers to conditions in which a population of organisms (e.g., host cells) is grown together and must compete for the same limited resources, for example, nutrients, oxygen, etc.

Host cells can be incubated in an environment in which there is no or little input of new nutrients. For example, host cells can be incubated in an environment in which there is no or little input of new oxygen, e.g., in sealed containers such as flasks.

Additionally or alternatively, host cells can be incubated in an culture medium that is well-mixed throughout the period of incubation, e.g., a shaking liquid culture. Generally, under such well-mixed conditions, the host cells have similar nutritional requirements and will be in competition for nutrients and/or oxygen (in the case of aerobic organisms) as the nutrients and/or oxygen become depleted by the growing population.

Additionally or alternatively, host cells can be incubated at an approximately constant temperature, e.g., at a temperature most suitable for the type of host cell. For example, for certain bacterial species including E. coli, host cells are typically incubated at a temperature that is around 37° C. In some embodiments, the host cells are incubated within 5° C., 4° C., 3° C., 2° C., or 1° C. of 37° C., e.g., at approximately 37° C.

Host cells can be incubated in a liquid culture that is shaken. This shaking is typically vigorous enough to prevent uneven distribution of nutrients and/or settling of some host cells at the bottom of the culture. For example, host cells can be shaken at at least 100 rpm (rotations per minute), at least 125 rpm, at least 150 rpm, at least 175 rpm, at least 200 rpm, at least 225 rpm, at least 250 rpm, at least 275 rpm, or at least 300 rpm. In some embodiments, host cells are shaken at between 100 rpm and 400 pm, e.g., between 200 and 350 rpm, e.g., at approximately 300 rpm.

Host cells can be incubated for a period of time before the plurality of bacteriophage is introduced into the culture. This period of time can allow, for example, the host cell population to recover from being in storage and/or to reach a particular ideal density before introduction of the plurality of bacteriophage. During this period of time before the plurality of bacteriophage is introduced, a selection pressure may be used, or it may not be used.

Culture conditions can comprise, e.g., continuous incubation of the host cells together with the bacteriophage over a period of time, e.g., at least 4 hours, at least 8 hours, at least 12 hours, or at least 16 hours. Additionally or alternatively, culture conditions can comprise continuous incubation of the host cells together with the bacteriophage until the growth of the host cells is saturated.

Culture conditions can allow continuous infection of the host cells by bacteriophage. That is, host cells are infect and re-infected continuously (if they survive) during the incubation period.

Additionally or alternatively, a selection pressure is introduced into the culture. For example, in particular with host cells transformed with exogenous DNA (such as plasmids), a selection pressure can be introduced to favor those host cells that maintain the exogenous DNA. Commonly used schemes include using one or more antibiotics as the selection pressure and a corresponding antibiotic resistance gene in the exogenous DNA that is to be maintained. This selection pressure may be the same as or different than that involving the selection agent as discussed herein, and, in some embodiments, both are used, e.g., sequentially and/or simultaneously.

In some embodiments, for at least a period of time during which transformed host cells are incubated together with bacteriophage, culture conditions include exposure to one or more antibiotics, to which some host cells may have resistance by virtue of an antibiotic resistance gene present on the phagemid, the polynucleotide in the library, or both. For example, both the phagemid and the polynucleotide in the library can have antibiotic resistance genes, e.g., the antibiotic resistance gene can be the same or different. If the phagemid contains one antibiotic resistance gene (a “first antibiotic resistance gene” conferring resistance to a “first antibiotic”) and the polynucleotide contains another antibiotic resistance gene (a “second antibiotic resistance gene” conferring resistance to a “second antibiotic”), culture conditions can comprise any of various schemes. As non-limiting examples, these conditions can comprise: 1) simultaneous exposure to both of the first antibiotic and the second antibiotic; 2) sequential exposure to the second antibiotic for a period of time (e.g., during a time period in which the host cells are incubated before bacteriophage are introduced into the culture), followed by exposure to either i) the first antibiotic or ii) both the first antibiotic and the second antibiotic (e.g., during a time period in which the host cells are incubated together with the bacteriophage); or 3) exposure to only one of the relevant antibiotics (e.g., the first antibiotic) during the course of the incubation.

### Selection Agents

Methods described herein can comprise a step of providing a plurality of bacteriophage comprising a phagemid encoding or serving as a template for a selection agent. Depending on the embodiment, the selection agent can confer either a survival advantage or a survival disadvantage to the host cell in the conditions in which the host cells are incubated with the bacteriophage. The selection agent can confer either an increase in cell growth kinetics or a decrease in cell growth kinetics to the host cell in the conditions in which the host cells are incubated with the bacteriophage.

The selection agent can be, e.g., a polypeptide and/or a polynucleotide.

In some embodiments, the selection agent confers a survival advantage to the host cell.

In some embodiments, the selection agent is encoded by a gene that is essential for survival of the host cell. Examples of such essential survival genes include, but are not limited to, genes involved in fatty acid biosynthesis; genes involved in amino acid biosynthesis; genes involved in cell division; genes involved in global regulatory functions; genes involved in protein translation and/or modification; genes involved in transcription; genes involved in protein degradation; genes encoding heat shock proteins; genes involved in ATP transport; genes involved in peptidoglycan synthesis; genes involved in DNA replication, repair, and/or modification; genes involved in tRNA modification and/or synthesis; and genes encoding ribosome components and/or involved in ribosome synthesis). For example, in Escherichia coli, a number of essential survival genes are known in the art, including, but not limited to, accD (acetylCoA carboxylase, carboxytransferase component, beta subunit), acpS (CoA:apo-[acyl-carrier-protein]pantetheinephosphotransferase), asd (aspartate-semialdehyde dehydrogenase), dapE (N-succinyl-diaminopimelate deacylase), dnaJ (chaperone with DnaK; heat shock protein), dnaK (chaperone Hsp70), era (GTP-binding protein), frr (ribosome releasing factor), ftsI (septum formation; penicillin-binding protein 3; peptidoglycan synthetase), ftsL cell division protein; ingrowth of wall at septum); ftsN (essential cell division protein); ftsZ (cell division; forms circumferential ring; tubulin-like GTP-binding protein and GTPase), gcpE, grpE (phage lambda replication; host DNA synthesis; heat shock protein; protein repair), hflB (degrades sigma32, integral membrane peptidase, cell division protein), infA (protein chain initiation factor IF-1), lgt (phosphatidylglycerol prolipoprotein diacylglyceryl transferase; a major membrane phospholipid), lpxC (UDP-3-O-acyl N-acetylglucosamine deacetylase; lipid A biosynthesis), map (methionine aminopeptidase), mopA (GroEL, chaperone Hsp60, peptide-dependent ATPase, heat shock protein), mopB (GroES, 10 Kd chaperone binds to Hsp60 in pres. Mg-ATP, suppressing its ATPase activity), msbA ATP-binding transport protein; multicopy suppressor of htrB), murA (first step in murein biosynthesis;UDP-N-glucosamine 1-carboxyvinyltransferase), murl (glutamate racemase, required for biosynthesis of D-glutamate and peptidoglycan), nadE (NAD synthetase, prefers NH3 over glutamine), nusG (component in transcription antitermination), parC (DNA topoisomerase IV subunit A), ppa (inorganic pyrophosphatase), proS (proline tRNA synthetase), pyrB (aspartate carbamoyltransferase, catalytic subunit), rpsB (30S ribosomal subunit protein S2), trmA (tRNA (uracil-5-)-methyltransferase), ycaH, ycfB, yfiL, ygjD (putative O-sialoglycoprotein endopeptidase), yhbZ (putative GTP-binding factor), yihA, and yjeQ. Additional essential genes in E. coli include those listed in “Experimental Determination and System-Level Analysis of Essential Genes in E. coli MG1655” by Gerdes 2003, e.g., in Supplementary Tables 1, 2, and 6.

In some embodiments, the selection agent is encoded by an antibiotic resistance gene, as discussed further below. For example, culture conditions can include exposure to the antibiotic to which the antibiotic resistance gene provides resistance.

In some embodiments, the selection agent inhibits a gene product that confers a survival disadvantage.

In certain embodiments, the selection agent confers a survival disadvantage to the host cell. The selection agent can be toxic to the host cell. For example, the selection agent can be a toxin, many of which are known in the art and many of which have been identified in various bacterial species. Examples of such toxins include, but are not limited to, ccdB, FlmA, fst, HicA, Hok, Ibs, Kid, LdrD, MazF, ParE, SymE, Tisb, TxpA/BrnT, XCV2162, yafO, Zeta and tse2. For example, the selection agent can be ccdB, which is found in E. coli. In other examples, the selection agent is tse2.

The selection agent can be toxic because it produces a toxic substance. For example, the production of the toxic substance can occur only in the presence of another agent, the presence of which may or may not be controlled externally.

Additionally or alternatively, the selection agent can inhibit a gene product that confers a survival advantage. By way of non-limiting example, the selection agent could be beta-lactamase inhibitory protein (BLIP), which inhibits beta-lactamases such as ampicillin and penicillin, among others.

### Induction Agents

Methods described herein can comprise a step of providing a library of polynucleotides, in which different polynucleotides in the library encode different versions of polypeptide of interest (or, in the case of a polynucleotide of interest, serve as a template for different version of the polynucleotide of interest). A polynucleotide can include, e .g., a regulatory element, e.g., promoter, which can control expression of the polypeptide. A regulatory element can be an inducible promoter, and expression can be induced by an induction agent. Such induction agent and/or induced expression can increase or improve the efficiency of selection.

The induction agent can be a polypeptide and/or a polynucleotide. The induction agent can also be a small molecule, light, temperature or an intracellular metabolite.

In some embodiments, the induction agents is arabinose, anhydrotetracycline, lactose, IPTG, propionate, blue light (470 nm) red light (650 nm), green light (532 nm) or L-rhamnose. For example, the induction agent can be arabinose.

### Polypeptides or Polynucleotides of Interest

In accordance with methods of the present disclosure, generally, the polypeptide and/or polynucleotide of interest binds to DNA.

For example, the polypeptide or polynucleotide of interest can be a polynucleotide that has DNA-binding ability, e.g., a DNA or RNA aptamer, a guide RNA (as discussed further herein, etc.).

Additionally or alternatively, the polypeptide or polynucleotide of interest can be a polypeptide of interest that is a DNA-binding protein or a variant thereof or comprises a portion that is a DNA-binding domain or a variant thereof.

The DNA-binding protein or domain can be site-specific. By “site-specific,” is meant that the DNA-binding protein or domain has a DNA sequence to which it preferentially binds, also known as a “recognition sequence.”

The polypeptide of interest can be a DNA-binding enzyme or comprises a DNA-binding domain thereof.

As a non-limiting example where the polypeptide of interest is a DNA-binding enzyme, the polypeptide of interest can comprise all or a portion of a site-specific recombinase. Non-limiting examples of site-specific recombinases include Cre recombinase, Flp recombinase, Dre recombinase, PhiC31 integrase, R recombinase, KD recombinase, B2 recombinase, B3 recombinase, gamma-delta serine recombinases, and Tn3 resolvase.

As another non-limiting example where the polypeptide of interest is a DNA-binding enzyme, the polypeptide of interest can comprise all or a portion of a nuclease, e.g., a site-specific nuclease.

The nuclease can be an endonuclease, e.g., a site-specific endonuclease (e.g., a restriction endonuclease, a meganuclease, a transcription activator-like effector nucleases (TALEN), a zinc finger nuclease, etc.).

Site specificity of a site-specific nuclease can be conferred by an accessory molecule. For example, the CRISPR-associated (Cas) nucleases are guided to specific sites by “guide RNAs” or gRNAs as described herein. In some embodiments, the nuclease is an RNA-guided nuclease. In some embodiments, the nuclease is a CRISPR-associated nuclease.

The nuclease can be a homolog or an ortholog of a previously known nuclease, for example, a newly discovered homolog or ortholog.

### RNA-Guided Nucleases

RNA-guided nucleases according to the present disclosure include, but are not limited to, naturally-occurring Class 2 CRISPR nucleases such as Cas9, and Cpf1, as well as other nucleases derived or obtained therefrom. For example, other nucleases derived or obtained therefrom include variant nucleases. In some embodiments, a variant nuclease comprises one or more altered enzymatic properties, e.g., altered nuclease activity or altered helicase activity (as compared with a naturally occurring or other reference nuclease molecule (including a nuclease molecule that has already been engineered or altered)). In some embodiments, a variant nuclease can have nickase activity or no cleavage activity (as opposed to double strand nuclease activity). In another embodiment, variant nucleases have an alteration that alters its size, e.g., a deletion of amino acid sequence that reduces its size, e.g., with or without significant effect on one or more, or any nuclease activity. In another embodiment, a variant nuclease can recognize a different PAM sequence. In some embodiments, a different PAM sequence is a PAM sequence other than that recognized by the endogenous wild-type PI domain of the reference nuclease, e.g., a non-canonical sequence.

In functional terms, RNA-guided nucleases are defined as those nucleases that: (a) interact with (e.g., complex with) a gRNA; and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif,” or “PAM,” which is described in greater detail below. RNA-guided nucleases can be defined, in broad terms, by their PAM specificity and cleavage activity, even though variations may exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g., Cas9 vs. Cpf1), species (e.g., S. pyogenes vs. S. aureus) or variation (e.g., full-length vs. truncated or split; naturally-occurring PAM specificity vs. engineered PAM specificity, etc.) of RNA-guided nuclease.

The PAM sequence takes its name from its sequential relationship to the “protospacer” sequence that is complementary to gRNA targeting domains (or “spacers”). Together with protospacer sequences, PAM sequences define target regions or sequences for specific RNA-guided nuclease/gRNA combinations.

Various RNA-guided nucleases may require different sequential relationships between PAMs and protospacers. In general, Cas9s recognize PAM sequences that are 3′ of the protospacer as visualized relative to the guide RNA targeting domain.

Cpf1, on the other hand, generally recognizes PAM sequences that are 5′ of the protospacer.

In addition to recognizing specific sequential orientations of PAMs and protospacers, RNA-guided nucleases can also recognize specific PAM sequences. S. aureus Cas9, for instance, recognizes a PAM sequence of NNGRRT or NNGRRV, wherein the N residues are immediately 3′ of the region recognized by the gRNA targeting domain. S. pyogenes Cas9 recognizes NGG PAM sequences. And F. novicida Cpf1 recognizes a TTN PAM sequence. PAM sequences have been identified for a variety of RNA-guided nucleases, and a strategy for identifying novel PAM sequences has been described by Shmakov et al., 2015, Molecular Cell 60, 385-397, Nov. 5, 2015. It should also be noted that engineered RNA-guided nucleases can have PAM specificities that differ from the PAM specificities of reference molecules (for instance, in the case of an engineered RNA-guided nuclease, the reference molecule may be the naturally occurring variant from which the RNA-guided nuclease is derived, or the naturally occurring variant having the greatest amino acid sequence homology to the engineered RNA-guided nuclease).

In addition to their PAM specificity, RNA-guided nucleases can be characterized by their DNA cleavage activity: naturally-occurring RNA-guided nucleases typically form DSBs in target nucleic acids, but engineered variants have been produced that generate only SSBs (discussed above) Ran & Hsu, et al., Cell 154(6), 1380-1389, September 12, 2013 (“Ran”), incorporated by reference herein), or that that do not cut at all.

Cas9

Crystal structures have been determined for S. pyogenes Cas9 (Jinek et al., Science 343(6176), 1247997, 2014 (“Jinek 2014”), and for S. aureus Cas9 in complex with a unimolecular guide RNA and a target DNA (Nishimasu 2014; Anders et al., Nature. 2014 Sep. 25; 513(7519):569-73 (“Anders 2014”); and Nishimasu 2015).

A naturally occurring Cas9 protein comprises two lobes: a recognition (REC) lobe and a nuclease (NUC) lobe; each of which comprise particular structural and/or functional domains. The REC lobe comprises an arginine-rich bridge helix (BH) domain, and at least one REC domain (e.g., a REC1 domain and, optionally, a REC2 domain). The REC lobe does not share structural similarity with other known proteins, indicating that it is a unique functional domain. While not wishing to be bound by any theory, mutational analyses suggest specific functional roles for the BH and REC domains: the BH domain appears to play a role in gRNA:DNA recognition, while the REC domain is thought to interact with the repeat:anti-repeat duplex of the gRNA and to mediate the formation of the Cas9/gRNA complex.

The NUC lobe comprises a RuvC domain, an HNH domain, and a PAM-interacting (PI) domain. The RuvC domain shares structural similarity to retroviral integrase superfamily members and cleaves the non-complementary (i.e., bottom) strand of the target nucleic acid. It may be formed from two or more split RuvC motifs (such as RuvC I, RuvCII, and RuvCIII in S. pyogenes and S. aureus). The HNH domain, meanwhile, is structurally similar to HNN endonuclease motifs, and cleaves the complementary (i.e., top) strand of the target nucleic acid. The PI domain, as its name suggests, contributes to PAM specificity.

While certain functions of Cas9 are linked to (but not necessarily fully determined by) the specific domains set forth above, these and other functions may be mediated or influenced by other Cas9 domains, or by multiple domains on either lobe. For instance, in S. pyogenes Cas9, as described in Nishimasu 2014, the repeat:antirepeat duplex of the gRNA falls into a groove between the REC and NUC lobes, and nucleotides in the duplex interact with amino acids in the BH, PI, and REC domains. Some nucleotides in the first stem loop structure also interact with amino acids in multiple domains (PI, BH and REC1), as do some nucleotides in the second and third stem loops (RuvC and PI domains).

Cpf1

The crystal structure of Acidaminococcus sp. Cpf1 in complex with crRNA and a double-stranded (ds) DNA target including a TTTN PAM sequence has been solved by Yamano et al. (Cell. 2016 May 5; 165(4): 949-962 (“Yamano”), incorporated by reference herein). Cpf1, like Cas9, has two lobes: a REC (recognition) lobe, and a NUC (nuclease) lobe. The REC lobe includes REC1 and REC2 domains, which lack similarity to any known protein structures. The NUC lobe, meanwhile, includes three RuvC domains (RuvC-I, -II and -III) and a BH domain. However, in contrast to Cas9, the Cpf1 REC lobe lacks an HNH domain, and includes other domains that also lack similarity to known protein structures: a structurally unique PI domain, three Wedge (WED) domains (WED-I, -II and -III), and a nuclease (Nuc) domain.

While Cas9 and Cpf1 share similarities in structure and function, it should be appreciated that certain Cpf1 activities are mediated by structural domains that are not analogous to any Cas9 domains. For instance, cleavage of the complementary strand of the target DNA appears to be mediated by the Nuc domain, which differs sequentially and spatially from the HNH domain of Cas9. Additionally, the non-targeting portion of Cpf1 gRNA (the handle) adopts a pseudoknot structure, rather than a stem loop structure formed by the repeat:antirepeat duplex in Cas9 gRNAs.

### Nucleic Acids Encoding RNA-Guided Nucleases

Nucleic acids encoding RNA-guided nucleases, e.g., Cas9, Cpf1 or functional fragments thereof, are provided herein. Exemplary nucleic acids encoding RNA-guided nucleases have been described previously (see, e.g., Cong et al., Science. 2013 Feb. 15; 339(6121):819-23 (“Cong 2013”); Wang et al., PLoS One. 2013 Dec. 31; 8(12):e85650 (“Wang 2013”); Mali 2013; Jinek 2012).

In some cases, a nucleic acid encoding an RNA-guided nuclease can be a synthetic nucleic acid sequence. For example, the synthetic nucleic acid molecule can be chemically modified. In certain embodiments, an mRNA encoding an RNA-guided nuclease will have one or more (e.g., all) of the following properties: it can be capped; polyadenylated; and substituted with 5-methylcytidine and/or pseudouridine.

Synthetic nucleic acid sequences can also be codon optimized, e.g., at least one non-common codon or less-common codon has been replaced by a common codon. For example, the synthetic nucleic acid can direct the synthesis of an optimized messenger mRNA, e.g., optimized for expression in a mammalian expression system, e.g., described herein. Examples of codon optimized Cas9 coding sequences are presented in WO 2016/073990 (“Cotta-Ramusino”).

In addition, or alternatively, a nucleic acid encoding an RNA-guided nuclease may comprise a nuclear localization sequence (NLS). Nuclear localization sequences are known in the art.

Guide RNA (gRNA) Molecules

The terms “guide RNA” and “gRNA” refer to any nucleic acid that promotes the specific association (or “targeting”) of an RNA-guided nuclease such as a Cas9 or a Cpf1 to a target sequence such as a genomic or episomal sequence in a cell. gRNAs can be unimolecular (comprising a single RNA molecule, and referred to alternatively as chimeric), or modular (comprising more than one, and typically two, separate RNA molecules, such as a crRNA and a tracrRNA, which are usually associated with one another, for instance by duplexing). gRNAs and their component parts are described throughout the literature, for instance in Briner et al. (Molecular Cell 56(2), 333-339, Oct. 23, 2014 (“Briner”), which is incorporated by reference), and in Cotta-Ramusino.

In bacteria and archea, type II CRISPR systems generally comprise an RNA-guided nuclease protein such as Cas9, a CRISPR RNA (crRNA) that includes a 5′ region that is complementary to a foreign sequence, and a trans-activating crRNA (tracrRNA) that includes a 5′ region that is complementary to, and forms a duplex with, a 3′ region of the crRNA. While not intending to be bound by any theory, it is thought that this duplex facilitates the formation of—and is necessary for the activity of—the Cas9/gRNA complex. As type II CRISPR systems were adapted for use in gene editing, it was discovered that the crRNA and tracrRNA could be joined into a single unimolecular or chimeric guide RNA, in one non-limiting example, by means of a four nucleotide (e.g., GAAA) “tetraloop” or “linker” sequence bridging complementary regions of the crRNA (at its 3′ end) and the tracrRNA (at its 5′ end). (Mali et al. Science. 2013 Feb. 15; 339(6121): 823-826 (“Mali 2013”); Jiang et al. Nat Biotechnol. 2013 March; 31(3): 233-239 (“Jiang”); and Jinek et al., 2012 Science August 17; 337(6096): 816-821 (“Jinek 2012”), all of which are incorporated by reference herein.)

Guide RNAs, whether unimolecular or modular, include a “targeting domain” that is fully or partially complementary to a target domain within a target sequence, such as a DNA sequence in the genome of a cell where editing is desired. Targeting domains are referred to by various names in the literature, including without limitation “guide sequences” (Hsu et al., Nat Biotechnol. 2013 September; 31(9): 827-832, (“Hsu”), incorporated by reference herein), “complementarity regions” (Cotta-Ramusino), “spacers” (Briner) and generically as “crRNAs” (Jiang). Irrespective of the names they are given, targeting domains are typically 10-30 nucleotides in length, and in certain embodiments are 16-24 nucleotides in length (for instance, 16, 17, 18, 19, 20, 21, 22, 23 or 24 nucleotides in length), and are at or near the 5′ terminus of in the case of a Cas9 gRNA, and at or near the 3′ terminus in the case of a Cpf1 gRNA.

In addition to the targeting domains, gRNAs typically (but not necessarily, as discussed below) include a plurality of domains that may influence the formation or activity of gRNA/Cas9 complexes. For instance, as mentioned above, the duplexed structure formed by first and secondary complementarity domains of a gRNA (also referred to as a repeat:anti-repeat duplex) interacts with the recognition (REC) lobe of Cas9 and can mediate the formation of Cas9/gRNA complexes. (Nishimasu et al., Cell 156, 935-949, February 27, 2014 (“Nishimasu 2014”) and Nishimasu et al., Cell 162, 1113-1126, August 27, 2015 (“Nishimasu 2015”), both incorporated by reference herein). It should be noted that the first and/or second complementarity domains may contain one or more poly-A tracts, which can be recognized by RNA polymerases as a termination signal. The sequence of the first and second complementarity domains are, therefore, optionally modified to eliminate these tracts and promote the complete in vitro transcription of gRNAs, for instance through the use of A-G swaps as described in Briner, or A-U swaps. These and other similar modifications to the first and second complementarity domains are within the scope of the present disclosure.

Along with the first and second complementarity domains, Cas9 gRNAs typically include two or more additional duplexed regions that are involved in nuclease activity in vivo but not necessarily in vitro. (Nishimasu 2015). A first stem-loop one near the 3′ portion of the second complementarity domain is referred to variously as the “proximal domain,” (Cotta-Ramusino) “stem loop 1” (Nishimasu 2014 and 2015) and the “nexus” (Briner). One or more additional stem loop structures are generally present near the 3′ end of the gRNA, with the number varying by species: S. pyogenes gRNAs typically include two 3′ stem loops (for a total of four stem loop structures including the repeat:anti-repeat duplex), while S. aureus and other species have only one (for a total of three stem loop structures). A description of conserved stem loop structures (and gRNA structures more generally) organized by species is provided in Briner.

While the foregoing description has focused on gRNAs for use with Cas9, it should be appreciated that other RNA-guided nucleases have been (or may in the future be) discovered or invented which utilize gRNAs that differ in some ways from those described to this point. For instance, Cpf1 (“CRISPR from Prevotella and Franciscella 1”) is a recently discovered RNA-guided nuclease that does not require a tracrRNA to function. (Zetsche et al., 2015, Cell 163, 759-771 Oct. 22, 2015 (“Zetsche I”), incorporated by reference herein). A gRNA for use in a Cpf1 genome editing system generally includes a targeting domain and a complementarity domain (alternately referred to as a “handle”). It should also be noted that, in gRNAs for use with Cpf1, the targeting domain is usually present at or near the 3′ end, rather than the 5′ end as described above in connection with Cas9 gRNAs (the handle is at or near the 5′ end of a Cpf1 gRNA).

Those of skill in the art will appreciate, however, that although structural differences may exist between gRNAs from different prokaryotic species, or between Cpf1 and Cas9 gRNAs, the principles by which gRNAs operate are generally consistent. Because of this consistency of operation, gRNAs can be defined, in broad terms, by their targeting domain sequences, and skilled artisans will appreciate that a given targeting domain sequence can be incorporated in any suitable gRNA, including a unimolecular or chimeric gRNA, or a gRNA that includes one or more chemical modifications and/or sequential modifications (substitutions, additional nucleotides, truncations, etc.). Thus, for economy of presentation in this disclosure, gRNAs may be described solely in terms of their targeting domain sequences.

More generally, skilled artisans will appreciate that some aspects of the present disclosure relate to systems, methods and compositions that can be implemented using multiple RNA-guided nucleases. For this reason, unless otherwise specified, the term gRNA should be understood to encompass any suitable gRNA that can be used with any RNA-guided nuclease, and not only those gRNAs that are compatible with a particular species of Cas9 or Cpf1. By way of illustration, the term gRNA can, in certain embodiments, include a gRNA for use with any RNA-guided nuclease occurring in a Class 2 CRISPR system, such as a type II or type V or CRISPR system, or an RNA-guided nuclease derived or adapted therefrom.

### Libraries of Polynucleotides Encoding Different Versions of a Cas9 Molecule

In some embodiments, methods and compositions of the present invention can be used with a library of polynucleotides that encode different versions of a Cas9 molecule or Cas9 polypeptide (e.g., a comprehensive and unbiased library of Cas9 mutants that span all or a portion of a Cas9 molecule or Cas9 polypeptide). In certain embodiments, methods and compositions of the present invention can be used to select one or more members of the library based on a particular property. In a typical embodiment, a Cas9 molecule or Cas9 polypeptide has the ability to interact with a gRNA molecule and, in concert with the gRNA molecule, localize to a site in a nucleic acid. Other activities, e.g., PAM specificity, cleavage activity, or helicase activity can vary more widely in Cas9 molecules and Cas9 polypeptides.

In some embodiments, methods and compositions of the present invention can be used to select one or more versions of a Cas9 molecule or Cas9 polypeptide which comprise altered enzymatic properties, e.g., altered nuclease activity or altered helicase activity (as compared with a naturally occurring or other reference Cas9 molecule including a Cas9 molecule that has already been engineered or altered). As discussed herein, a mutated version of a reference Cas9 molecule or Cas9 polypeptide can have nickase activity or no cleavage activity (as opposed to double strand nuclease activity). In an embodiment, methods and compositions of the present invention can be used to select one or more versions of a Cas9 molecule or Cas9 polypeptide which have an alteration that alters its size, e.g., a deletion of amino acid sequence that reduces its size, e.g., with or without significant effect on one or more, or any Cas9 activity. In an embodiment, methods and compositions of the present invention can be used to select one or more versions of a Cas9 molecule or Cas9 polypeptide which recognizes a different PAM sequence (e.g., a version of a Cas9 molecule can be selected to recognize a PAM sequence other than that recognized by the endogenous wild-type PI domain of the reference Cas9 molecule).

Libraries with different versions of a Cas9 molecule or Cas9 polypeptide can be prepared using any method, e.g., by alteration of a parental, e.g., naturally occurring, Cas9 molecules or Cas9 polypeptides, to provide a library of altered Cas9 molecules or Cas9 polypeptides. For example, one or more mutations or differences relative to a parental Cas9 molecule, e.g., a naturally occurring or engineered Cas9 molecule, can be introduced. Such mutations and differences comprise: substitutions (e.g., conservative substitutions or substitutions of non-essential amino acids); insertions; or deletions. In an embodiment, a Cas9 molecule or Cas9 polypeptide in a library of the present invention can comprise one or more mutations or differences, e.g., at least 1, 2, 3, 4, 5, 10, 15, 20, 30, 40 or 50 mutations but less than 200, 100, or 80 mutations relative to a reference, e.g., a parental, Cas9 molecule.

### Libraries of Guide RNA Molecules

In some embodiments, methods and compositions of the present disclosure can be used with a library of guide RNA molecules and/or polynucleotides encoding guide RNA molecules. For example, a library can be provided and/or generated that includes DNA molecules that each encodes a guide RNA having (i) a different targeting domain described herein; (ii) different first and/or secondary complementarity domains described herein; and/or (iii) a different stem loop described herein. As described herein, a library can be introduced into a host cell. In some embodiments, a nucleic acid encoding an RNA-guided nuclease, e.g., a Cas9 molecule or Cas9 polypeptide, is also introduced into the host cell.

In certain embodiments, methods and compositions of the present disclosure can be used to select one or more members of the guide RNA library based on a particular property, such as ability to localize to a site in a nucleic acid and/or to interact with a Cas9 molecule or Cas9 polypeptide and/or to localize a Cas9 molecule or Cas9 polypeptide to a site in a nucleic acid.

Libraries with different versions of a guide RNA can be prepared using any method, e.g., by alteration of a parental, e.g., naturally occurring, guide RNA, to provide a library of altered guide RNAs. For example, one or more mutations or differences relative to a parental guide RNA, e.g., a naturally occurring or engineered guide RNA, can be introduced. Such mutations and differences comprise: substitutions; insertions; or deletions. In some embodiments, a guide RNA in a library of the present disclosure can comprise one or more mutations or differences, e.g., at least 1, 2, 3, 4, 5, 10, 15, 20, 30, 40 or 50 mutations but less than 200, 100, or 80 mutations relative to a reference, e.g., a parental, guide RNA.

### Transcription Regulator

In some embodiments, the polypeptide of interest is a transcription regulator (e.g., a transcription factor) or comprises a DNA-binding domain thereof. In some embodiments, the polypeptide of interest is a transcriptional activator. In some embodiments, the polypeptide of interest is a transcriptional repressor.

### Fusions

In some embodiments, the polypeptide of interest is a fusion protein that is or comprises at least two heterologous domains, one of which is a DNA-binding domain as discussed further herein. In some embodiments, the DNA-binding domain is a Cas9. In some embodiments, the Cas9 comprises a nuclease inactive domain.

In some embodiments, one of the heterologous domains is a site-specific recombinase. In some embodiments, the polypeptide of interest is a fusion protein that is or comprises a DNA-binding domain and all or a portion of a site-specific recombinase. Site-specific recombinases are known in the art. Non-limiting examples of site-specific recombinases include Cre recombinase, Flp recombinase, Dre recombinase, PhiC31 integrase, R recombinase, KD recombinase, B2 recombinase, B3 recombinase, gamma-delta serine recombinases, and Tn3 resolvase.

In some embodiments, one of the heterologous domains is a nucleic-acid editing domain. In some embodiments, the polypeptide of interest is a fusion protein that is or includes a DNA-binding domain and all or a portion of a nucleic-acid editing domain. Nucleic-acid editing domains are known in the art. Non-limiting examples of nucleic-acid editing domains include, e.g., a DNA-editing domain and a deaminase domain. Deaminases include, e.g., a cytidine deaminase, an apolipoprotein B mRNA-editing complex (APOBEC) family deaminase, an APOBEC1 family deaminase, an activation-induced cytidine deaminase (AID), an ACF1/ASE deaminase, an adenosine deaminase, and an ADAT family deaminase. In some embodiments, the polypeptide of interest is a fusion protein that is or includes a nucleic-acid-editing domain at the N-terminus and a Cas9 domain at the C-terminus. In some embodiments, the polypeptide of interest is a fusion protein that is or includes a nucleic-acid-editing domain at the C terminus and a Cas9 domain at the N-terminus. In some embodiments, the fusion protein includes a linker between the Cas9 domain and the nucleic acid-editing domain.

Methods of generating polynucleotides that encode fusion polypeptides are known in the art.

Nuclease Fusions
For example, in some embodiments in which the polypeptide of interest is a nuclease, the nuclease comprises a DNA binding domain fused to a heterologous DNA cleavage domain. The heterologous DNA cleavage domain can be a cleavage domain from any nuclease. As non-limiting examples, the heterologous DNA cleavage domain can be a cleavage domain from a restriction endonuclease or a FokI nuclease domain.

Examples of nucleases comprising a DNA binding domain fused to a heterologous DNA cleavage domain include, but are not limited to, zinc finger nucleases, TALENs, and mega-TALEs.

In some embodiments, the nuclease comprises an enzymatically inactive Cas protein fused to a heterologous DNA cleavage domain.

Transcriptional Regulator Fusions

For example, in some embodiments in which the polypeptide of interest is a transcription regulator, the polypeptide of interest comprises a DNA binding domain fused to a heterologous transcriptional regulation domain, e.g., a transcriptional activator or repressor. One or more than one copy of a transcriptional regulation domain can be included, e.g., multiple copies of the same transcriptional regulation domain and/or combinations of different transcriptional regulation domains.

For example, in some embodiments, a polypeptide of interest is selected based on the ability to bind to a DNA target site. In some embodiments, the selection scheme is based on the transcriptional activation of a gene that confers a survival advantage to the host cell. In some embodiments, the selection scheme is based on the transcriptional repression of a gene that confers a survival disadvantage to the host cell.

For example, in some embodiments, a polypeptide of interest is selected based on the inability to bind to a DNA target site. In some embodiments, the selection scheme is based on the transcriptional repression of a gene that confers a survival advantage to the host cell. In some embodiments, the selection scheme is based on the transcriptional activation of a gene that confers a survival disadvantage to the host cell.

Any of a variety of transcriptional regulation domains can be used in accordance with methods of the invention. Generally, the type of transcriptional regulation domain used may be chosen based on factors such as the host cell type, degree of sensitivity desired in the system, target gene (that is to be activated and/or repressed), etc.

In some embodiments, the transcriptional regulation domain is a transcriptional activation domain. Non-limiting examples of suitable domains for transcriptional activation include the VP16 activation domain from herpes simplex virus, nuclear hormone receptors, the p65 subunit of nuclear factor kappa B, artificial chimeric functional domains such as VP64, OsGAI, HALF-1, C1, AP1, ARF-5, ARF-6, ARF-7, ARF-8, CPRF1, CPRF4, MYC-RP/GP, and TRAB1. In some embodiments, the transcriptional regulation domain is VP16 or VP64.

In some embodiments, the transcriptional regulation domain is a transcriptional repression domain. Non-limiting examples of suitable domains for transcriptional repression include, but are not limited to, KRAB A, KRAB B, KOX, TGF-beta-inducible early gene (TIEG), v-erbA, SID, MBD2, MBD3, DNMT1, DNMT3A, DNMT3B, other members of the Dnmt (DNA methyltransferase) family, Rb, MeCP, ROM2 and AtHD2A. In some embodiments, the transcriptional regulation domain is KRAB A, KRAB B or KOX.

### DNA-Binding Domains

The DNA-binding domain can be a DNA-binding domain of any of DNA-binding protein, such as any of the DNA-binding proteins mentioned herein. As non-limiting examples, the DNA-binding domain can comprises a TALE DNA binding domain, a zinc finger DNA binding domain, a meganuclease DNA binding domain, or an enzymatically inactive Cas protein.

In some embodiments, the DNA-binding domain is an enzymatically inactive Cas protein. In some embodiments, the DNA-binding domain is an enzymatically inactive Cas9 protein.

### DNA Target Sites

In general, the DNA target site for a particular inventive method may depend on the physical location of the DNA target site (e.g., in some aspects the DNA target site may be located on a phagemid while in other aspects the DNA target site may be located within the host cell genome), the nature of the polypeptide or polynucleotide of interest, the nature of the selection process and/or the desired outcome of the selection process. DNA target sites can be located within a variety of types of nucleotide sequences. For example, in some embodiments, the DNA target site may be located within an element that is not transcribed, within an element that encodes a polypeptide or serves as a template for a polynucleotide (e.g., a non-coding RNA), within a regulatory element that controls expression of a polypeptide, etc.

As described herein, in some embodiments, the DNA target site may be located on a phagemid. In some embodiments, the DNA target site may be located on a plasmid. In situations where the selection process relies on cleavage (or non-cleavage) of the phagemid, or plasmid, the DNA target site can be located anywhere on the phagemid, or plasmid, since selection relies on linearization (and subsequent destruction) of the phagemid, or plasmid, which may result from cleavage at any position on the phagemid, or plasmid. In situations where the selection process relies on repression (or activation) of expression of a selection agent, the DNA target site may be located within a regulatory element that drives expression of the selection agent. In some embodiments, the regulatory element may be an inducible regulatory element.

As describe herein, in some embodiments, the DNA target site may be located within a host cell genome. In situations where the selection process relies on cleavage of an endogenous gene that is essential for survival of the host cell (an “essential gene”), the DNA target site can, for example, be located within the coding or regulatory elements of the essential gene. In situations where the selection process relies on repression of an essential gene, the DNA target site may be at any location in the host cell genome that leads to repression of the essential gene when bound by the polypeptide of interest (e.g., within a regulatory element of the essential gene, between the promoter and coding region of the essential gene, etc.).

The specific nucleotide sequence of the DNA target site (i.e., separate and apart from whether it is located on a phagemid or within a host cell genome) will generally depend on the nature of the polypeptide of interest, the nature of the selection process and the desired outcome of the selection process. By way of example, when the polypeptide of interest is a reference nuclease (e.g., a meganuclease, TALEN or zinc finger nuclease) that recognizes a first nucleotide sequence and the inventive methods are being used to select for one or more modified versions of the reference nuclease that selectively bind a second nucleotide sequence which differs from the first nucleotide sequence (e.g., at 1, 2, 3, etc. bases) then the inventive methods may involve using a DNA target site which corresponds to the second nucleotide sequence in a positive selection step and a DNA target site which corresponds to the first nucleotide sequence in a negative selection step (i.e., to select for versions of the reference nuclease that bind the second nucleotide sequence but do not bind the first nucleotide sequence).

In the case of Cas molecules (e.g., Cas9 molecules) the DNA target site will be determined in part based on the PAM of the Cas molecule and the sequence of the targeting domain of the gRNA which is used to localize the Cas molecule at the DNA target site. By way of example, when the polypeptide of interest is a reference Cas9 molecule that recognizes a first PAM sequence and the inventive methods are being used to select for one or more modified versions of the reference Cas9 molecule that selectively recognize a second PAM sequence which differs from the first PAM sequence (e.g., at 1, 2, 3, etc. bases) then the inventive methods may involve using a DNA target site which includes the second PAM sequence in a positive selection step and a DNA target site which includes the first PAM sequence in a negative selection step (i.e., to select for versions of the reference Cas9 molecule that recognize the second PAM sequence but do not recognize the first PAM sequence). In both cases the DNA target site will also include a sequence that is complementary to the sequence of the targeting domain of the gRNA which is used to localize the Cas9 molecule at the DNA target site.

In some embodiments, methods provided herein can be used for evaluation of the ability of PAM variants to direct cutting of a target site by an RNA-guided nuclease, e.g., a variant S. pyogenes Cas9.

In some embodiments, the library comprises a plurality of nucleic acid templates which further include nucleotide sequences comprising PAM variants adjacent to the target site. In some embodiments, a PAM sequence comprises the sequence NGA, NGAG, NGCG, NNGRRT, NNGRRA or NCCRRC.

Some of the methods provided herein allow for the simultaneous assessment of a plurality of PAM variants for any given target site, and in some embodiments, in combination with a variant S. pyogenes Cas9. Accordingly, data obtained from such methods can be used to compile a list of PAM variants that mediate cleaving of a particular target site in combination with wild-type S. pyogenes Cas9 or a variant S. pyogenes Cas9. In some embodiments, a sequencing method is used to generate quantitative sequencing data, and relative abundance of cleavage of a particular target site mediated by a particular PAM variant can be determined.

### Antibiotic Resistance Genes

In certain embodiments, plasmids in the library and/or phagemids comprise an antibiotic resistance gene.

In some embodiments, the antibiotic resistance gene confers resistance to an antibiotic that kills or inhibits the growth of bacteria such as E. coli. Non-limiting examples of such antibiotics include ampicillin, bleomycin, carbenicillin, chloramphenicol, erythromycin, kanamycin, penicillin, polymyxin B, spectinomycin, streptomycin, and tetracycline. A variety of antibiotic resistance gene cassettes are known and available in the art and/or are commercially available, e.g., as elements in plasmids. For example, there are a number of commercially available plasmids with ampR (ampicillin resistance), bleR (bleomycin resistance), carR (carbenicillin resistance), cmR (chloramphenicol resistance), kanR (kanamycin resistance), and/or tetR (tetracycline resistance) or gene elements. An additional example of an antibiotics resistance gene is beta-lactamase.

In some embodiments, phagemids comprise a first antibiotic resistance gene and plasmids in the library comprise a second antibiotic resistance gene. In some embodiments, the first antibiotic resistance gene is distinct from the second antibiotic resistance gene. For example, in some embodiments, the first antibiotic resistance gene is a cmR (chloramphenicol resistance) gene, and the second antibiotic resistance gene is an ampR (ampicillin resistance) gene.

### Regulatory Elements

In certain embodiments, gene elements (such as, for example, those encoding selection agents, antibiotic resistance genes, polypeptide, polynucleotides etc.) are operably linked to regulatory elements to allow expression of one or more other elements, e.g., selection agents, antibiotic resistance genes, polypeptides, polynucleotides etc.

In some embodiments, the phagemid includes a regulatory element that drives expression of one or more gene elements on the phagemid, for example, the selection agent.

In some embodiments, polynucleotides in the library include a regulatory element that drives expression of one or more gene elements on the polynucleotide, for example, the polypeptide or polynucleotide of interest, and, if present, a gene element encoding a selection agent such as an antibiotic resistance gene.

A wide variety of gene regulatory elements exist. The type of regulatory element used can depend, for example, on the host cell, the type of gene intended to be expressed, other factors such as transcription factors that are used, etc.

Gene regulatory elements include, but are not limited to, enhancers, promoters, operators, terminators, etc., as well as combinations thereof. As a non-limiting example, a regulatory element can comprise both a promoter and an operator.

In some embodiments, the regulatory element is constitutive in that it is active in all circumstances in the cell. For example, a constitutive element such as a constitutive promoter can be used to express a gene product without requiring additional regulation.

In some embodiments, the regulatory element is inducible, i.e., it is only active in response to a specific stimulus.

For example, the lac operator is inducible in that it can be made active in the presence of IPTG (Isopropyl β-D-1-thiogalactopyranoside). Another example, is the arabinose promoter that is made active in the presence of arabinose.

In some embodiments, the regulatory element is bidirectional, in that it can drive expression of a gene placed on other side of it in a sequence. Thus, in some embodiments, expression of at least two gene elements can be driven by the same gene element.

Gene segments that serve as regulatory elements are readily available in the art, and many are commercially available from vendors. For example, expression plasmids or other vectors that already contain one or more regulatory elements to express a gene segment of interest are readily available.

Analysis of Selected Versions of Polypeptides and/or Polynucleotides

After one or more rounds of selection, selected versions of polypeptides and/or polynucleotides can be recovered from host cells that survived the selection and analyzed. In schematics using more than one cycle of evolution (mutagenesis followed by one or more selection rounds), this analysis can happen at the end of every cycle or only in some cycles.

Examples of types of analysis include, but are not limited to, sequencing, binding and/or cleavage assays (including in vitro assays), verification of activity of selected versions in cell types other than the host cell type.

As a non-limiting example, next generation (also known as high throughput sequencing) can be performed to sequence all or most of the selected variants.

In some embodiments, deep sequencing is performed, meaning that each nucleotide is read several times during the sequencing process, for example at a depth of greater than at least 7, at least 10, at least 15, at least 20, or ever greater, wherein depth (D) is calculated as

D=N×L/G (Equation 1),

wherein N is the number of reads, L is the length of the original genome, and G is length of the polynucleotide being sequenced.

In some embodiments, Sanger sequencing is used to analyze at least some of the selected versions.

Analysis of the sequences may be used, for example, to check for enriched amino acid residues or nucleotide, which are indicative of selected versions.

Alternatively or additionally, a sample of selected versions may be sequenced, e.g., from individual host cell colonies (e.g., bacterial colonies).

Binding and/or cleavage assays are known in the art. Some of these assays are performed in vitro, e.g., using cell components or isolated molecules (such as polypeptides, polynucleotides, or ribonuclear proteins) rather than whole cells.

In some embodiments, an in vitro assay for binding and/or cleavage of a DNA substrate is performed. In some embodiments, the assay tests the activity of lysates extracted from host cells that survived one or more rounds of selection. In some embodiments, the assay tests the activity of polypeptides, polynucleotides, and/or ribonuclear proteins, or complexes thereof, extracted from host cells that survived one or more rounds of selection.

In some embodiments, analysis comprises performing one or more assays to test one or more function(s) of the products of the selected versions of polynucleotides in the library (e.g., polypeptides encoded by the selected version or polynucleotides whose template is the selected version).

### Uses

In some embodiments, selection methods of the present invention are used together with a mutagenesis method that generates the library of plasmids. Any mutagenesis method can be used with selection methods of the present invention.

In some embodiments, one round of mutagenesis followed by one or more rounds of selection is used. This cycle may be performed once, or it may be repeated one or more times, e.g., as part of a directed evolution strategy, in which the versions of polypeptides and/or polynucleotides of interest that are selected in one cycle are mutagenized in the mutagenesis round of the next cycle. Cycles can be repeated as many times as desired, for example, until the selected versions of the polypeptide and/or polynucleotide of interest obtained meet certain criteria and/or a desired number of selected polypeptides and/or polynucleotides meeting certain criteria are obtained.

In some embodiments, in one cycle, one round of mutagenesis is followed by a round of positive selection (e.g., for versions of a polypeptide and/or polynucleotide of interest that cleave and/or bind a DNA target site).

In some embodiments, in one cycle, one round of mutagenesis is followed by a round of positive selection, which is followed by a round of negative selection (e.g., for versions of a polypeptide and/or polynucleotide of interest that do not cleave and/or do not bind a DNA target site).

In some embodiments, in one cycle, one round of mutagenesis is followed by a round of negative selection, which is followed by a round of positive selection.

In embodiments in which more than one cycle is performed, the cycles need not have the same schematic in terms of mutagenesis and selection rounds. Additionally, other details need not be the same between cycles, for example, the method of mutagenesis need not be the same from one cycle to the next, nor do the exact conditions or schematics of the selection rounds need to be the same.

Accordingly, selection methods of the present disclosure can be used to select for polypeptides and/or polynucleotides of interest with desired binding and/or cleaving site specificities.

For example, selection methods can be used to select for polypeptides and/or polynucleotides of interest that bind to one allele but not another allele. For example, the ability to discriminate between a disease allele and a wild-type allele can be used to develop therapies, for example, based on gene editing, gene repression, and/or gene activation techniques. In some embodiments, for example, a positive selection is carried out to select for polypeptides or polynucleotides of interest that recognize one allele (e.g., a disease allele), and then a negative selection is a carried out to select against polypeptides or polynucleotides of interest that recognize another allele (e.g., a wild-type allele). In some embodiments, a negative selection is carried out to select against polypeptides or polynucleotides of interest that recognize one allele (e.g., a wild type allele), then a positive selection is carried out to select for polypeptides and/or polynucleotides of interest that recognize the other allele (e.g., a disease allele).

As illustrated in the Examples, selection methods of the present invention have been used in evolution schemes to evolve a polypeptide with the ability to discriminate between alleles differing by only one base change.

As another example, selection methods can be used to select for polypeptides and/or polynucleotides of interest that have altered binding preferences, e.g., as compared to naturally occurring polypeptides and/or polynucleotides of interest. For example, certain DNA-binding proteins (including enzymes) have very limited binding specificities, therefore limiting their uses. Selecting for and/or evolving site-specific DNA-binding domains or proteins with altered binding specificities (e.g., as compared to that of naturally occurring polypeptides and/or polynucleotides of interest) may increase the range of their use.

In some embodiments, a positive selection is carried out to select for polypeptides and/or polynucleotides of interest that recognize one DNA target site (e.g., a desired new target site), and then a negative selection is a carried out to select against polypeptides and/or polynucleotides of interest that recognize another DNA target site (e.g., the native target site).

In some embodiments, a positive selection is carried out to select for polypeptides and/or polynucleotides of interest that recognize one DNA target site (e.g., a desired new target site), and no negative selection is a carried out.

In some embodiments, a negative selection is carried out to select against polypeptides and/or polynucleotides of interest that recognize one DNA target site (e.g., the native target site), then a positive selection is carried out to select for polypeptides and/or polynucleotides of interest that recognize another DNA target site (e.g., a desired new target site).

As another example, selection methods can be used to select for polypeptides or polynucleotides of interest with reduced off-target activity. Although certain DNA-binding proteins are classified as specific for a particular recognition sequence, some may exhibit promiscuity in that they bind to some degree to one or more off-target sites.

In some embodiments, for example, a negative selection is carried out to select for polypeptides or polynucleotides of interest that do not recognize one or more off-target sites. When it is desired to select against more than one off-target site, in some embodiments, a pool of bacteriophage containing different phagemids is used, wherein each of the different phagemids contains a DNA target site corresponding to one of the off-target sites. Because host cells can be infected again and again by various bacteriophage during the incubating step, it is possible to select against binding to or cleaving at multiple off-targets in a single round of negative selection.

In some embodiments, for example, a positive selection is carried out to select for polypeptides or polynucleotides of interest that recognize a particular recognition sequence, and then a negative selection is a carried out to select against polypeptides or polynucleotides of interest that recognize one or more off-target sites. In some embodiments, a negative selection is carried out to select against polypeptides or polynucleotides of interest that recognize one or more off-target sites, then a positive selection is carried out to select for polypeptides or polynucleotides of interest that recognize a particular recognition sequence.

In some embodiments in which more than one round of selection is used (e.g., a positive selection round and then a negative selection round) in one cycle, methods comprise a step of pelleting (e.g., by centrifugation) the host cells in between rounds of selection. Such a pelleting step may, for example, remove agents used during a previous selection round (e.g., antibiotics, inducers of gene expression such as IPTG, etc.).

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure is further illustrated by the following examples. The examples are provided for illustrative purposes only. They are not to be construed as limiting the scope or content of the disclosure in any way.

### EXAMPLES

### Example 1

### Evolution of an Allele-Specific Cas9 to a Single Base-Pair Mutation Conferring Cone Rod Dystrophy 6 (CORD6)

The present Example demonstrates that selection methods of the present invention can be used in an evolution strategy to evolve a site-specific nuclease with specificity for a disease allele differing only by a point mutation (a single base change) as compared to the wild type, non-disease allele.

The use of Cas9 or other targeted nucleases in allele-specific cutting of heterozygous sequences is hindered by promiscuous activity, especially with alleles differing by a single base. We aimed to engineer Cas9 mutants which could selectively cut only one allele, here selectively cutting alleles with the R838S mutation in the retinal guanylate cyclase (GUCY2D) protein, which confers the CORD6 disease phenotype. We constructed plasmid pEvol_CORD6, which encodes a Cas9 protein and a gRNA targeting the CORD6 sequence TAACCTGGAGGATCTGATCC (SEQ ID NO: 15). pEvol_CORD6 also constitutively expresses beta-lactamase, which confer resistance to ampicillin. Two phagemids (plasmids containing phage origin f1 elements), pSelect_CORD6 and pSelect_GUCY2DWT, were also constructed, containing potential target sites TAACCTGGAGGATCTGATCCGGGAGA (SEQ ID NO: 16) and TAACCTGGAGGATCTGATCCGGGAGC (SEQ ID NO: 17), respectively. Bold bases indicate the site of the R838S mutation. The site of the mutation was chosen to be targeted to the sixth position of the wild-type Cas9 PAM (NNGRRT). In this example, we selected for Cas9 mutants that cut adjacent to a modified PAM with an A in the sixth position (i.e., NNGRRA) (“positive selection”) while also selecting against Cas9 mutants that cut adjacent to a modified PAM with a C in the sixth position (i.e., NNGRRC) (“negative selection”).

pSelect_CORD6 and pSelect_GUCY2DWT also each contain a constitutively expressed chloramphenicol resistance gene and ccdB (a bacterial toxin) under the control of lac promoter, which allows induction of ccdB expression by IPTG (Isopropyl β-D-1-thiogalactopyranoside).

pSelect_CORD6 and pSelect_GUCY2DWT were separately packaged into helper bacteriophage.

To engineer allele specificity, two E. coli bacterial libraries of Cas9 mutants were generated using the pEvol_CORD6 plasmid as the initial template for mutagenesis, using a comprehensive and unbiased mutagenesis method that targeted every codon and allowed tuning of the mutation rate. One library was tuned such that it had a median of 3 amino acid mutations per Cas9 polypeptide (“low” mutation rate), the other had a median of 5 amino acid mutations per Cas9 polypeptide (“high” mutation rate).

In each round of evolution, we subjected each bacterial library of pEvol_CORD6 mutants first to a positive selection for cutting against phage containing pSelect_CORD6, and then to a negative selection against cutting pSelect_GUCY2DWT, in a competitive culture with continuous challenge by phage as follows:

To infect bacteria, phage packaging the appropriate pSelect plasmid was added to saturated bacteria containing a library of pEvol_CORD6 mutants, and the bacterial library was cultured in ampicillin in a liquid culture. For each library, the entire library was cultured in the same liquid culture.

After this initial incubation and infection, positive selection was carried out by adding 1 mM IPTG, which induces ccdB. Cultures were then grown overnight, e.g., for at least 12 hours. Cells were then pelleted, which removes some IPTG. Negative selection was then carried out by growing the bacteria in the presence of 50 μg/ml chloramphenicol (which is constitutively expressed by both pSelect plasmids) and absence of IPTG during a second overnight culture. During both positive and negative selection, bacteria were continuously infected by phage present in the liquid culture, thus presenting a continuous challenge to either cut (in the case of positive selection) or not cut (in the case of negative selection).

Pooled plasmid DNA from all selected library members following negative selection was used as templates for the next mutagenesis reaction. We repeated three rounds of mutagenesis (which generates libraries), positive selection, and negative selection in this manner. By applying dual selection pressures on each library, stringent selection was performed for a Cas9 mutant that contained a PAM specific to the CORD6 allele.

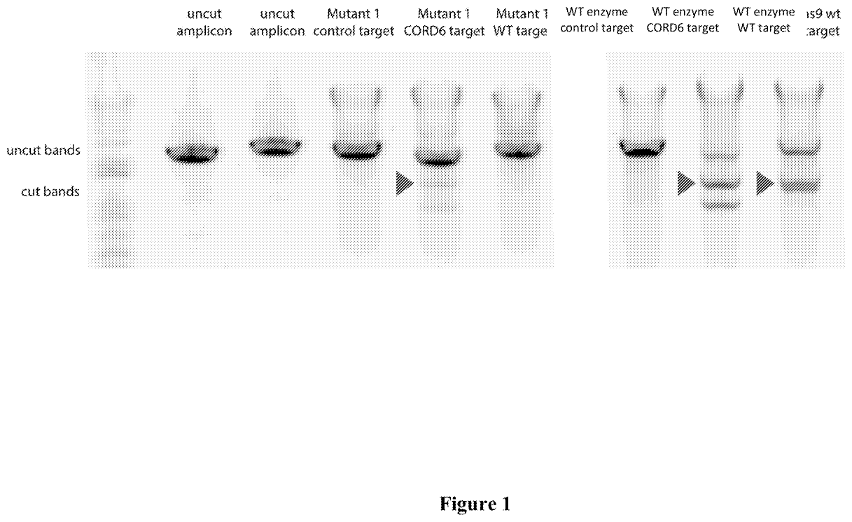

PacBio next-generation sequencing on plasmid DNA isolated from the pooled selected library members was performed in every evolution cycle, after the negative selection round. After only the first evolution cycle, we found that a particular mutant accounted for about 20% of the population, indicating high selective strength. We proceeded to test the cleavage activity of this mutant using E. coli cell lysate containing the mutant protein on amplicons either containing the wildtype or mutated GUCY2D sequence. We observed cleavage only on the CORD6 amplicons (FIG. 1). Further analysis of the PAM preference of this mutant also indicated two-fold higher specificity for the sixth-position A rather than C. The activity of this highly selected mutant confirms the designed selective pressures and demonstrates successful engineering of an allele-specific Cas9 mutant through an unbiased mutagenesis method and a competitive selection strategy.

### Example 2

### Evolution of Cas9 with Reduced Off-Target Activities Using Known Off-Targets

The present Example describes how selection methods of the present invention can be used in an evolution strategy to reduce off-target activity of a site-specific DNA-binding enzyme.

Off-target cleavage is a common byproduct of Cas9 targeted DNA cleavage. In order to mitigate this effect, selection for on-target cleavage (“positive selection”) can be coupled with selection against known or potential off-target sequences (“negative selection”) in our system. Off-targets, such as those discovered by GUIDE-SEQ or other methods, can be counter-selected in an informed manner. Alternatively, libraries of potential off-targets, such as single-base-pair mismatches, can be selected against. In this way, specific guides can be tailored to preferentially cleave at the appropriate site by combining them with a Cas9 that has been evolved to reduce off-target cleavage.

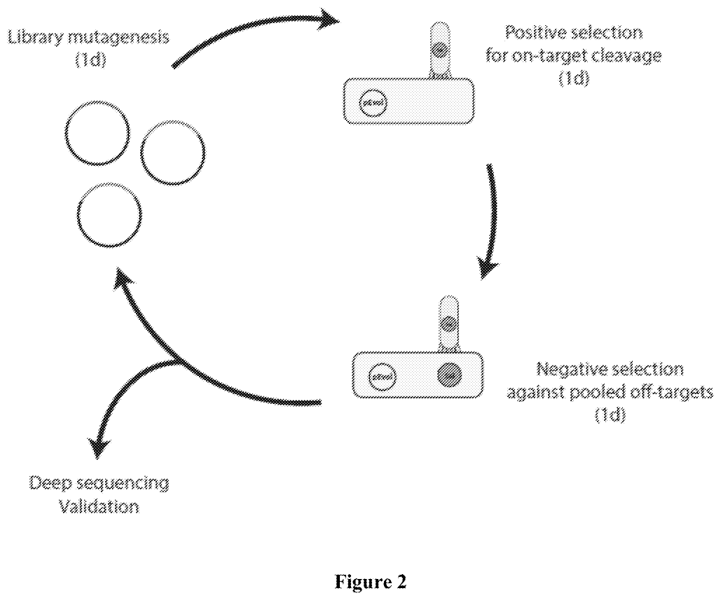

Evolution in this case proceeds by first selecting for cleavage of the on-target in positive selection and then for a negative selection against mixed phage populations of the designated off-targets followed by optional deep sequencing validation (FIG. 2). This evolutionary algorithm may be repeated over several rounds.

### Example 3

### Evolution of an Allele-Specific Cas9

The present Example demonstrates that selection methods of the present invention can be used in an evolution strategy to evolve a site-specific nuclease with specificity for a disease allele differing only by a point mutation (a single base change) as compared to the wild type, non-disease allele. However, the selection methods of the present invention may also be used to evolve a site-specific nuclease with specificity for an allele (e.g., a mutant or disease allele) differing by greater than a single base change as compared to another allele (e.g, wild-type or non-disease allele).

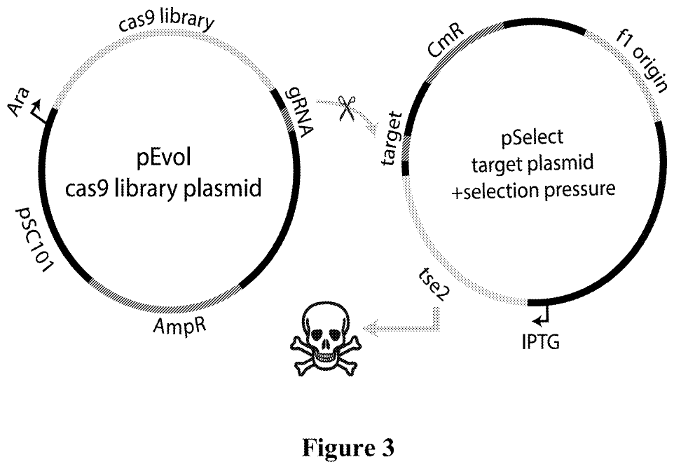

The use of Cas9 or other targeted nucleases in allele-specific cutting of heterozygous sequences is hindered by promiscuous activity, especially with alleles differing by a single base. We aimed to engineer Cas9 mutants which would selectively cut only one allele (e.g., allele 1, mutant allele), and not cut an allele differing by a single base (e.g., allele 2, wild-type allele). We also aimed to improve the efficiency of methods that select for Cas9 mutants to achieve the greatest discrimination for the cutting of one allele. Selection of the most discriminating Cas9 mutants may be achieved by control of, for example, the amount of Cas9 present in the selection process and/or, improvement in the efficiency of the positive and negative selection. The amount of Cas9 in a selection system may be controlled by, for example, use of lower copy numbers of a plasmid which expresses Cas9. In some embodiments, amount of Cas9 is controlled by placing expression of Cas9 under the control of an inducible promoter. In some embodiments, an inducible promoter is an arabinose promoter (FIG. 3).

Positive and negative selection processes may rely on inducible expression of toxin molecules and/or expression of resistance to a drug such as an antibiotic. For example, when expression of a toxin is induced from a plasmid, only cells which comprise a Cas9 mutant that recognizes and cuts an appropriate target (e.g., allele 1, mutant allele) in the plasmid will survive. Cells which comprise a Cas9 that does not recognize and cut the appropriate target, are killed by the toxin. This positive selection step selects for all Cas9 molecules that are capable of recognizing and cutting the appropriate target (e.g., allele 1, mutant allele).

In another embodiment, when cells are treated with an antibiotic, only cells which comprise a Cas9 that cuts an inappropriate target in a plasmid conferring resistance to the antibiotic are killed. Cells which comprise a Cas9 that does not recognize an inappropriate target (e.g., allele 2, wild type allele) maintain resistance to the antibiotic and survive. This negative selection step selects against Cas9 molecules that are capable of recognizing and cutting the inappropriate target (e.g., allele 2, wild type allele).

Utility of positive and negative selection steps for the identification of highly selective Cas9 molecules relies, at least in part, on a high degree of discrimination in cell killing. Comparison of cell growth kinetics during selection can characterize the efficiency of the selection for optimal Cas9 molecules.

Efficiency of Selection Using tse2

A plasmid, pEvol_CAS, which encodes a Cas9 protein and a gRNA targeting a target sequence was constructed. A plasmid, pEvol_NONTARGETING, which encodes a Cas9 protein and a non-targeting gRNA was also constructed. Both plasmids constitutively expresses beta-lactamase, which confer resistance to ampicillin (AmpR) and an inducible arabinose promoter (Ara) to control expression of Cas9. Phagemids (plasmid containing phage origin f1 elements), pSelect_MUT and pSelect_WT were also constructed, each containing a potential target site. The phagemids also contained a constitutively expressed chloramphenicol resistance gene (CmR) and tse2 (a bacterial toxin) under the control of lac promoter, which allows induction of tse2 expression by IPTG (Isopropyl β-D-1-thiogalactopyranoside). pSelect_MUT and pSelect_WT were each separately packaged into helper bacteriophage.

To engineer allele specificity, two E. coli bacterial libraries of Cas9 mutants were generated using the pEvol_CAS plasmid as the initial template for mutagenesis, using a comprehensive and unbiased mutagenesis method that targeted every codon and allowed tuning of the mutation rate. One library was tuned such that it had a median of 3 amino acid mutations per Cas9 polypeptide (“low” mutation rate), the other had a median of 5 amino acid mutations per Cas9 polypeptide (“high” mutation rate).

In each round of evolution, we subjected each bacterial library of pEvol_CAS mutants to positive selection for cutting against phage containing pSelect_MUT in a competitive culture with continuous challenge by phage as follows:

To infect bacteria, phage packaging the pSelect_MUT plasmid was added to bacteria containing a library of pEvol_CAS mutants or pEvol_NONTARGETING, and the bacterial library was cultured in ampicillin in a liquid culture. For each library, the entire library was cultured in the same liquid culture.

After this initial incubation and infection, the stringency of positive selection using tse2 was assessed by adding 1 mM IPTG, to induce tse2 expression, to a subset of the pEvol_CAS cultures and to a subset of the pEvol_NONTARGETING cultures. Expression of Cas9 and guide RNA was induced by addition of arabinose. Cas9 and guide RNA expression was not induced in a subset of the pEvol_CAS cultures that were treated with IPTG. Cultures were then grown overnight, e.g., for at least 12 hours. During positive selection, bacteria were continuously infected by phage present in the liquid culture, thus presenting a continuous challenge to cut the target.

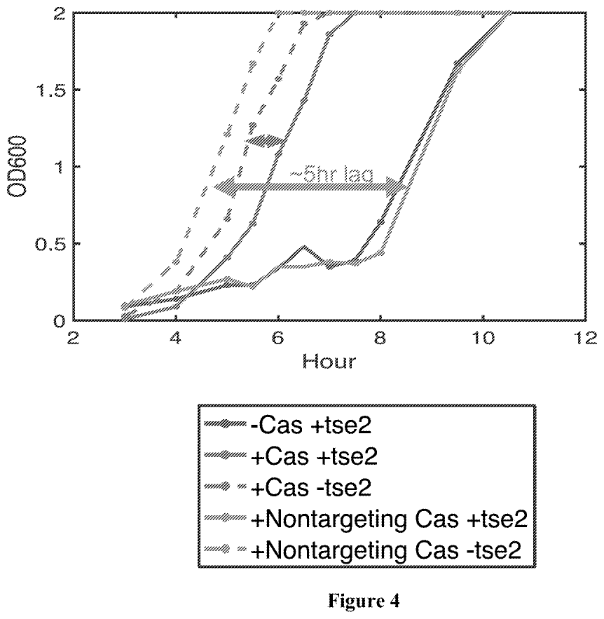

As shown in FIG. 4, cultures expressing tse2 but not Cas9 (−Cas +tse2) or expressing a nontargeting guide RNA (+Nontargeting Cas +tse2) exhibited a significant growth lag due to induction of tse2. In comparison, cultures induced to express tse2, but also expressing a Cas9 and targeting guide RNA (+Cas +tse2), which would be expected to cut the target and suppress expression of tse2, demonstrated a rapid growth over approximately 7 hours. Cultures which expressed Cas9 and either a targeting or non-targeting guide RNA, but were not induced to express tse2, demonstrated rapid cell growth over the first 6 hours. These data demonstrate that tse2 has significant cell killing effect when no Cas9 is present, or when the guide RNA does not recognize the target. These data also demonstrate that appropriately targeted Cas9 and guide RNA off-set the effects of induction of tse2 expression.

### Efficiency of Selection by Modulating Cas9 Expression

A plasmid library, pEvol_CASLIBRARY, was generated using the pEvol_WTCAS plasmid as the initial template for mutagenesis and a comprehensive and unbiased mutagenesis method that targeted every codon and allowed tuning of the mutation rate. The plasmids encode a Cas9 protein and a gRNA targeting a target sequence. A plasmid pEvol_WTCAS, which encodes a wild-type Cas9 protein and a targeting gRNA, was also constructed. Both plasmids constitutively expresses beta-lactamase, which confer resistance to ampicillin (AmpR) and an inducible arabinose promoter (Ara) to control expression of Cas9. Phagemids (plasmid containing phage origin f1 elements), pSelect_MUT and pSelect_WT were also constructed, containing potential target sites, as described above.

To infect bacteria, phage packaging the pSelect_MUT plasmid was added to saturated bacteria containing a library of pEvol_CASLIBRARY mutants or pEvol_WTCAS, and the bacterial library was cultured in ampicillin in a liquid culture. For each library, the entire library was cultured in the same liquid culture.

After this initial incubation and infection, the stringency of positive selection using tse2 and wild-type Cas or the Cas library was assessed by adding 1 mM IPTG, to induce tse2 expression, to a subset of the pEvol_CASLIBRARY cultures and to a subset of the pEvol_WTCAS cultures. Expression of Cas9 and gRNA was induced by addition of arabinose. Cas9 and gRNA expression was not induced in a subset of the pEvol_CASLIBRARY and pEvol_WT CAS cultures that were treated with IPTG. Cultures were then grown overnight, e.g., for at least 12 hours. During positive selection, bacteria were continuously infected by phage present in the liquid culture, thus presenting a continuous challenge to cut the target.

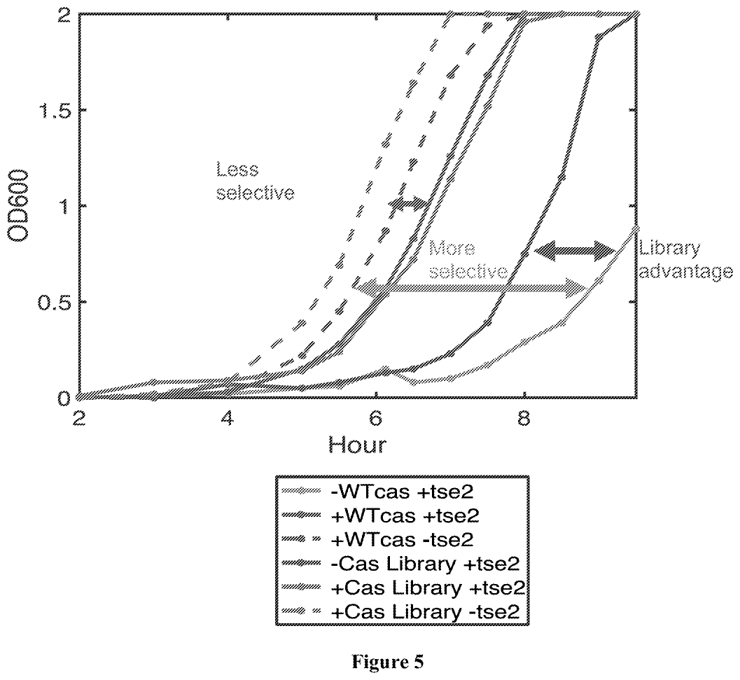

As shown in FIG. 5, cultures expressing tse2 but neither wild-type Cas9 (−WTCas +tse2) or a mutant Cas9 library (−Cas Library+tse2) exhibited a significant growth lag due to induction of tse2. However, wild-type Cas9 cultures exhibited a greater growth lag than mutant Cas9 library cultures indicating leaky expression of Cas9 mutants, even in the absence of arabinose. In comparison, cultures induced to express tse2, but also expressing a Cas9 and targeting guide RNA (+WTcas +tse2 or +Cas Library +tse2), which would be expected to cut the target and suppress expression of tse2, demonstrated rapid growth over approximately 7 hours. Cultures which expressed either wild-type Cas9 or Cas9 library mutants, but were not induced to express tse2, demonstrated rapid cell growth over the first 6 hours. The difference in cell growth between cultures expressing wild-type Cas9, with or without tse2, was less than the difference in cell growth between cultures expressing Cas9 library mutants, with or without tse2. These data suggest that Cas9 library mutants exhibit greater cutting activity than wild-type Cas9. These data also confirmed that tse2 has significant cell killing effect when no Cas9 is present.

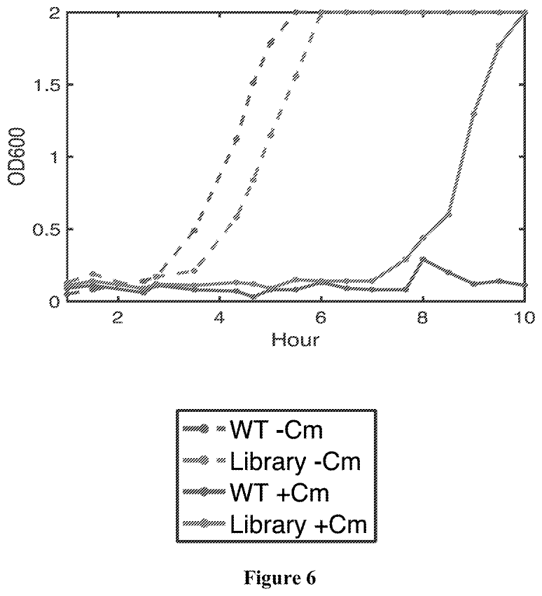

Negative selection was also carried out by growing the bacteria in the presence of 50 μg/ml chloramphenicol (resistance to chloramphenicol is constitutively expressed by the pSelect_MUT and pSelect_WT phagemids) during an overnight culture. Control cultures were not treated with chloramphenicol. During negative selection, bacteria were continuously infected by phage present in the liquid culture, thus presenting a continuous challenge to cut the appropriate target (allele 1, mutant allele) and to not cut the inappropriate target (allele 2, wild-type allele). Both wild-type Cas9 (WTCas +Cm) and mutant Cas9 library (Library+Cm) exhibited a significant growth lag due to elimination of resistant to chloramphenicol by off-target cutting (FIG. 6). However, mutant Cas9 library mutants demonstrated recovery in growth due to selection of Cas9 mutants that did not exhibit off-target cutting and maintained chloramphenicol resistance.

### Library Evolution

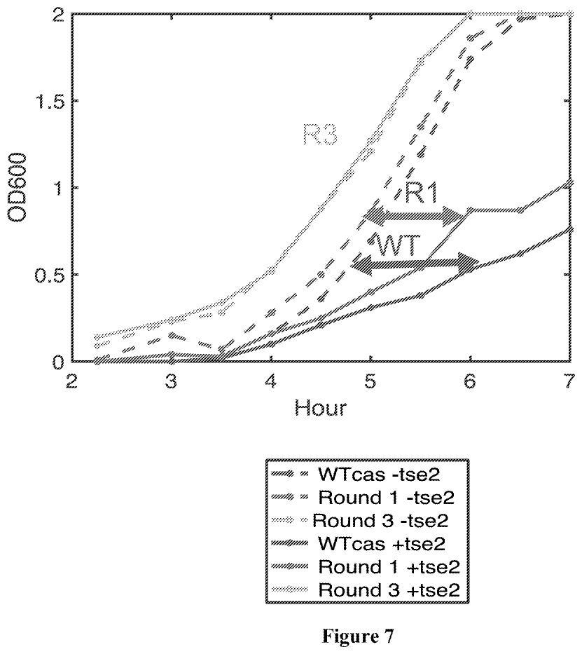

Successive rounds of library evolution generated Cas9 mutants with high levels of selectivity for cutting a target. This is demonstrated by successive reduction in the growth lag when cultures are induced to express tse2. FIG. 7 shows a significant growth lag for wild-type Cas9 cultures when tse2 is induced (WTcas +tse2) and a significant negative delta when compared to growth of cultures expressing wild-type Cas9 without induction of tse2 (WTcas −tse2). The delta in cell growth is reduced following one round of mutagenesis (for example, Round 1 +tse2 versus Round 1 −tse2). Following three rounds of mutagenesis cell growth curves are nearly identical for cultures induced to express tse2 and those that have not been induced to express tse2. These data indicate that use of tse2 and selective rounds of mutagenesis can generate a mutant Cas9 that this highly selective for on-target cutting.

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

SEQUENCE LISTING

An exemplary codon optimized nucleic acid sequence encoding a Cas9 molecule
of S. pyogenes (SEQ ID NO: 3).

| | |
|---|---|
| atggataaaa agtacagcat cgggctggac atcggtacaa actcagtggg gtgggccgtg | 60 |
| attacggacg agtacaaggt accctccaaa aaatttaaag tgctgggtaa cacggacaga | 120 |
| cactctataa agaaaaatct tattggagcc ttgctgttcg actcaggcga gacagccgaa | 180 |
| gccacaaggt tgaagcggac cgccaggagg cggtatacca ggagaaagaa ccgcatatgc | 240 |
| tacctgcaag aaatcttcag taacgagatg gcaaaggttg acgatagctt tttccatcgc | 300 |
| ctggaagaat cctttcttgt tgaggaagac aagaagcacg aacggcaccc catctttggc | 360 |
| aatattgtcg acgaagtggc atatcacgaa aagtacccga ctatctacca cctcaggaag | 420 |
| aagctggtgg actctaccga taaggcggac ctcagactta tttatttggc actcgcccac | 480 |
| atgattaaat ttagaggaca tttcttgatc gagggcgacc tgaacccgga caacagtgac | 540 |
| gtcgataagc tgttcatcca acttgtgcag acctacaatc aactgttcga agaaaaccct | 600 |
| ataaatgctt caggagtcga cgctaaagca atcctgtccg cgcgcctctc aaaatctaga | 660 |
| agacttgaga atctgattgc tcagttgccc ggggaaaaga aaaatggatt gtttggcaac | 720 |
| ctgatcgccc tcagtctcgg actgacccca aatttcaaaa gtaacttcga cctggccgaa | 780 |
| gacgctaagc tccagctgtc caaggacaca tacgatgacg acctcgacaa tctgctggcc | 840 |
| cagattgggg atcagtacgc cgatctcttt ttggcagcaa agaacctgtc cgacgccatc | 900 |
| ctgttgagcg atatcttgag agtgaacacc gaaattacta aagcacccct tagcgcatct | 960 |
| atgatcaagc ggtacgacga gcatcatcag gatctgaccc tgctgaaggc tcttgtgagg | 1020 |
| caacagctcc ccgaaaaata caaggaaatc ttctttgacc agagcaaaaa cggctacgct | 1080 |
| ggctatatag atggtggggc cagtcaggag gaattctata aattcatcaa gcccattctc | 1140 |
| gagaaaatgg acggcacaga ggagttgctg gtcaaactta acagggagga cctgctgcgg | 1200 |
| aagcagcgga cctttgacaa cgggtctatc ccccaccaga ttcatctggg cgaactgcac | 1260 |
| gcaatcctga ggaggcagga ggatttttat ccttttctta aagataaccg cgagaaaata | 1320 |
| gaaaagattc ttacattcag gatcccgtac tacgtgggac ctctcgcccg gggcaattca | 1380 |
| cggtttgcct ggatgacaag gaagtcagag gagactatta caccttggaa cttcgaagaa | 1440 |
| gtggtggaca agggtgcatc tgcccagtct ttcatcgagc ggatgacaaa ttttgacaag | 1500 |
| aacctcccta atgagaaggt gctgcccaaa cattctctgc tctacgagta ctttaccgtc | 1560 |
| tacaatgaac tgactaaagt caagtacgtc accgagggaa tgaggaagcc ggcattcctt | 1620 |
| agtggagaac agaagaaggc gattgtagac ctgttgttca agaccaacag gaaggtgact | 1680 |
| gtgaagcaac ttaaagaaga ctactttaag aagatcgaat gttttgacag tgtggaaatt | 1740 |
| tcaggggttg aagaccgctt caatgcgtca ttggggactt accatgatct tctcaagatc | 1800 |
| ataaaggaca aagacttcct ggacaacgaa gaaaatgagg atattctcga agacatcgtc | 1860 |
| ctcaccctga ccctgttcga agacagggaa atgatagaag agcgcttgaa aacctatgcc | 1920 |
| cacctcttcg acgataaagt tatgaagcag ctgaagcgca ggagatacac aggatgggga | 1980 |
| agattgtcaa ggaagctgat caatggaatt agggataaac agagtggcaa gaccatactg | 2040 |
| gatttcctca aatctgatgg cttcgccaat aggaacttca tgcaactgat tcacgatgac | 2100 |
| tctcttacct tcaaggagga cattcaaaag gctcaggtga gcgggcaggg agactccctt | 2160 |
| catgaacaca tcgcgaattt ggcaggttcc cccgctatta aaaagggcat ccttcaaact | 2220 |
| gtcaaggtgg tggatgaatt ggtcaaggta atgggcagac ataagccaga aaatattgtg | 2280 |
| atcgagatgg cccgcgaaaa ccagaccaca cagaagggcc agaaaaatag tagagagcgg | 2340 |
| atgaagagga tcgaggaggg catcaaagag ctgggatctc agattctcaa agaacacccc | 2400 |
| gtagaaaaca cacagctgca gaacgaaaaa ttgtacttgt actatctgca gaacggcaga | 2460 |
| gacatgtacg tcgaccaaga acttgatatt aatagactgt ccgactatga cgtagaccat | 2520 |
| atcgtgcccc agtccttcct gaaggacgac tccattgata acaaagtctt gacaagaagc | 2580 |
| gacaagaaca ggggtaaaag tgataatgtg cctagcgagg aggtggtgaa aaaaatgaag | 2640 |
| aactactggc gacagctgct taatgcaaag ctcattacac aacggaagtt cgataatctg | 2700 |
| acgaaagcag agagaggtgg cttgtctgag ttggacaagg cagggtttat taagcggcag | 2760 |
| ctggtggaaa ctaggcagat cacaaagcac gtggcgcaga ttttggacag ccggatgaac | 2820 |
| acaaaatacg acgaaaatga taaactgata cgagaggtca aagttatcac gctgaaaagc | 2880 |
| aagctggtgt ccgattttcg gaaagacttc cagttctaca aagttcgcga gattaataac | 2940 |
| taccatcatg ctcacgatgc gtacctgaac gctgttgtcg ggaccgcctt gataaagaag | 3000 |
| tacccaaagc tggaatccga gttcgtatac ggggattaca aagtgtacga tgtgaggaaa | 3060 |
| atgatagcca agtccgagca ggagattgga aaggccacag ctaagtactt cttttattct | 3120 |
| aacatcatga atttttttaa gacggaaatt accctggcca acggagagat cagaaagcgg | 3180 |
| ccccttatag agacaaatgg tgaaacaggt gaaatcgtct gggataaggg cagggatttc | 3240 |
| gctactgtga ggaaggtgct gagtatgcca caggtaaata tcgtgaaaaa aaccgaagta | 3300 |
| cagaccggag gattttccaa ggaaagcatt ttgcctaaaa gaaactcaga caagctcatc | 3360 |
| gcccgcaaga aagattggga ccctaagaaa tacgggggat ttgactcacc caccgtagcc | 3420 |
| tattctgtgc tggtggtagc taaggtggaa aaaggaaagt ctaagaagct gaagtccgtg | 3480 |
| aaggaactct tgggaatcac tatcatggaa agatcatcct ttgaaaagaa ccctatcgat | 3540 |
| ttcctggagg ctaagggtta caaggaggtc aagaaagacc tcatcattaa actgccaaaa | 3600 |
| tactctctct tcgagctgga aaatggcagg aagagaatgt tggccagcgc cggagagctg | 3660 |
| caaaagggaa acgagcttgc tctgccctcc aaatatgtta attttctcta tctcgcttcc | 3720 |
| cactatgaaa agctgaaagg gtctcccgaa gataacgagc agaagcagct gttcgtcgaa | 3780 |
| cagcacaagc actatctgga tgaaataatc gaacaaataa gcgagttcag caaaagggtt | 3840 |
| atcctggcgg atgctaattt ggacaaagta ctgtctgctt ataacaagca ccgggataag | 3900 |
| cctattaggg aacaagccga gaatataatt cacctcttta cactcacgaa tctcggagcc | 3960 |
| cccgccgcct tcaaatactt tgatacgact atcgaccgga aacggtatac cagtaccaaa | 4020 |
| gaggtcctcg atgccaccct catccaccag tcaattactg gcctgtacga aacacggatc | 4080 |
| gacctctctc aactgggcgg cgactag | 4107 |

An exemplary codon optimized nucleic acid sequences encoding a Cas9 molecule
of S. aureus (SEQ ID NO: 7).

| | |
|---|---|
| atgaaaagga actacattct ggggctggac atcgggatta caagcgtggg gtatgggatt | 60 |
| attgactatg aaacaaggga cgtgatcgac gcaggcgtca gactgttcaa ggaggccaac | 120 |
| gtggaaaaca atgagggacg gagaagcaag aggggagcca ggcgcctgaa acgacggaga | 180 |
| aggcacagaa tccagagggt gaagaaactg ctgttcgatt acaacctgct gaccgaccat | 240 |
| tctgagctga gtggaattaa tccttatgaa gccagggtga aaggcctgag tcagaagctg | 300 |
| tcagaggaag agttttccgc agctctgctg cacctggcta agcgccgagg agtgcataac | 360 |
| gtcaatgagg tggaagagga caccggcaac gagctgtcta caaaggaaca gatctcacgc | 420 |
| aatagcaaag ctctggaaga gaagtatgtc gcagagctgc agctggaacg gctgaagaaa | 480 |
| gatggcgagg tgagagggtc aattaatagg ttcaagacaa gcgactacgt caaagaagcc | 540 |
| aagcagctgc tgaaagtgca gaaggcttac caccagctgg atcagagctt catcgatact | 600 |
| tatatcgacc tgctggagac tcggagaacc tactatgagg gaccaggaga agggagcccc | 660 |
| ttcggatgga aagacatcaa ggaatggtac gagatgctga tgggacattg cacctatttt | 720 |
| ccagaagagc tgagaagcgt caagtacgct tataacgcag atctgtacaa cgccctgaat | 780 |
| gacctgaaca acctggtcat caccagggat gaaaacgaga aactggaata ctatgagaag | 840 |
| ttccagatca tcgaaaacgt gtttaagcag aagaaaaagc ctacactgaa acagattgct | 900 |
| aaggagatcc tggtcaacga agaggacatc aagggctacc gggtgacaag cactggaaaa | 960 |
| ccagagttca ccaatctgaa agtgtatcac gatattaagg acatcacagc acggaaagaa | 1020 |
| atcattgaga acgccgaact gctggatcag attgctaaga tcctgactat ctaccagagc | 1080 |
| tccgaggaca tccaggaaga gctgactaac ctgaacagcg agctgaccca ggaagagatc | 1140 |
| gaacagatta gtaatctgaa ggggtacacc ggaacacaca acctgtccct gaaagctatc | 1200 |
| aatctgattc tggatgagct gtggcataca aacgacaatc agattgcaat ctttaaccgg | 1260 |
| ctgaagctgg tcccaaaaaa ggtggacctg agtcagcaga aagagatccc aaccacactg | 1320 |
| gtggacgatt tcattctgtc acccgtggtc aagcggagct tcatccagag catcaaagtg | 1380 |
| atcaacgcca tcatcaagaa gtacggcctg cccaatgata tcattatcga gctggctagg | 1440 |
| gagaagaaca gcaaggacgc acagaagatg atcaatgaga tgcagaaacg aaaccggcag | 1500 |
| accaatgaac gcattgaaga gattatccga actaccggga aagagaacgc aaagtacctg | 1560 |
| attgaaaaaa tcaagctgca cgatatgcag gagggaaagt gtctgtattc tctggaggcc | 1620 |
| atccccctgg aggacctgct gaacaatcca ttcaactacg aggtcgatca tattatcccc | 1680 |
| agaagcgtgt ccttcgacaa ttcctttaac aacaaggtgc tggtcaagca ggaagagaac | 1740 |
| tctaaaaagg gcaataggac tcctttccag tacctgtcta gttcagattc caagatctct | 1800 |
| tacgaaacct ttaaaaagca cattctgaat ctggccaaag gaaagggccg catcagcaag | 1860 |
| accaaaaagg agtacctgct ggaagagcgg gacatcaaca gattctccgt ccagaaggat | 1920 |
| tttattaacc ggaatctggt ggacacaaga tacgctactc gcggcctgat gaatctgctg | 1980 |
| cgatcctatt tccgggtgaa caatctggat gtgaaagtca agtccatcaa cggcgggttc | 2040 |
| acatcttttc tgaggcgcaa atggaagttt aaaaaggagc gcaacaaagg gtacaagcac | 2100 |
| catgccgaag atgctctgat tatcgcaaat gccgacttca tctttaagga gtggaaaaag | 2160 |
| ctggacaaag ccaagaaagt gatggagaac cagatgttcg aagagaagca ggccgaatct | 2220 |
| atgcccgaaa tcgagacaga acaggagtac aaggagattt tcatcactcc tcaccagatc | 2280 |
| aagcatatca aggatttcaa ggactacaag tactctcacc gggtggataa aaagcccaac | 2340 |
| agagagctga tcaatgacac cctgtatagt acaagaaaag acgataaggg gaataccctg | 2400 |
| attgtgaaca atctgaacgg actgtacgac aaagataatg acaagctgaa aaagctgatc | 2460 |
| aacaaaagtc ccgagaagct gctgatgtac caccatgatc ctcagacata tcagaaactg | 2520 |
| aagctgatta tggagcagta cggcgacgag aagaacccac tgtataagta ctatgaagag | 2580 |
| actgggaact acctgaccaa gtatagcaaa aaggataatg gccccgtgat caagaagatc | 2640 |
| aagtactatg ggaacaagct gaatgcccat ctggacatca cagacgatta ccctaacagt | 2700 |
| cgcaacaagg tggtcaagct gtcactgaag ccatacagat tcgatgtcta tctggacaac | 2760 |
| ggcgtgtata aatttgtgac tgtcaagaat ctggatgtca tcaaaaagga gaactactat | 2820 |
| gaagtgaata gcaagtgcta cgaagaggct aaaaagctga aaaagattag caaccaggca | 2880 |
| gagttcatcg cctcctttta caacaacgac ctgattaaga tcaatggcga actgtatagg | 2940 |
| gtcatcgggg tgaacaatga tctgctgaac cgcattgaag tgaatatgat tgacatcact | 3000 |
| taccgagagt atctggaaaa catgaatgat aagcgccccc ctcgaattat caaaacaatt | 3060 |
| gcctctaaga ctcagagtat caaaaagtac tcaaccgaca ttctgggaaa cctgtatgag | 3120 |
| gtgaagagca aaaagcaccc tcagattatc aaaaagggc | 3159 |

An exemplary codon optimized nucleic acid sequences encoding a Cas9 molecule
of S. aureus (SEQ ID NO: 8).

| | |
|---|---|
| atgaagcgga actacatcct gggcctggac atcggcatca ccagcgtggg ctacggcatc | 60 |
| atcgactacg agacacggga cgtgatcgat gccggcgtgc ggctgttcaa agaggccaac | 120 |
| gtggaaaaca acgagggcag gcggagcaag agaggcgcca gaaggctgaa gcggcggagg | 180 |
| cggcatagaa tccagagagt gaagaagctg ctgttcgact acaacctgct gaccgaccac | 240 |
| agcgagctga gcggcatcaa cccctacgag gccagagtga agggcctgag ccagaagctg | 300 |
| agcgaggaag agttctctgc cgccctgctg cacctggcca agagaagagg cgtgcacaac | 360 |
| gtgaacgagg tggaagagga caccggcaac gagctgtcca ccaaagagca gatcagccgg | 420 |
| aacagcaagg ccctggaaga gaaatacgtg gccgaactgc agctggaacg gctgaagaaa | 480 |
| gacggcgaag tgcggggcag catcaacaga ttcaagacca gcgactacgt gaaagaagcc | 540 |
| aaacagctgc tgaaggtgca gaaggcctac caccagctgg accagagctt catcgacacc | 600 |
| tacatcgacc tgctggaaac ccggcggacc tactatgagg gacctggcga gggcagcccc | 660 |
| ttcggctgga aggacatcaa agaatggtac gagatgctga tgggccactg cacctacttc | 720 |
| cccgaggaac tgcggagcgt gaagtacgcc tacaacgccg acctgtacaa cgccctgaac | 780 |
| gacctgaaca atctcgtgat caccagggac gagaacgaga agctggaata ttacgagaag | 840 |
| ttccagatca tcgagaacgt gttcaagcag aagaagaagc ccaccctgaa gcagatcgcc | 900 |
| aaagaaatcc tcgtgaacga agaggatatt aagggctaca gagtgaccag caccggcaag | 960 |
| cccgagttca ccaacctgaa ggtgtaccac gacatcaagg acattaccgc ccggaaagag | 1020 |
| attattgaga acgccgagct gctggatcag attgccaaga tcctgaccat ctaccagagc | 1080 |
| agcgaggaca tccaggaaga actgaccaat ctgaactccg agctgaccca ggaagagatc | 1140 |
| gagcagatct ctaatctgaa gggctatacc ggcacccaca acctgagcct gaaggccatc | 1200 |
| aacctgatcc tggacgagct gtggcacacc aacgacaacc agatcgctat cttcaaccgg | 1260 |
| ctgaagctgg tgcccaagaa ggtggacctg tcccagcaga aagagatccc caccaccctg | 1320 |
| gtggacgact tcatcctgag ccccgtcgtg aagagaagct tcatccagag catcaaagtg | 1380 |
| atcaacgcca tcatcaagaa gtacggcctg cccaacgaca tcattatcga gctggcccgc | 1440 |
| gagaagaact ccaaggacgc ccagaaaatg atcaacgaga tgcagaagcg gaaccggcag | 1500 |
| accaacgagc ggatcgagga aatcatccgg accaccggca aagagaacgc caagtacctg | 1560 |
| atcgagaaga tcaagctgca cgacatgcag gaaggcaagt gcctgtacag cctggaagcc | 1620 |
| atccctctgg aagatctgct gaacaacccc ttcaactatg aggtggacca catcatcccc | 1680 |
| agaagcgtgt ccttcgacaa cagcttcaac aacaaggtgc tcgtgaagca ggaagaaaac | 1740 |
| agcaagaagg gcaaccggac cccattccag tacctgagca gcagcgacag caagatcagc | 1800 |
| tacgaaacct tcaagaagca catcctgaat ctggccaagg gcaagggcag aatcagcaag | 1860 |
| accaagaaag agtatctgct ggaagaacgg gacatcaaca ggttctccgt gcagaaagac | 1920 |
| ttcatcaacc ggaacctggt ggataccaga tacgccacca gaggcctgat gaacctgctg | 1980 |
| cggagctact tcagagtgaa caacctggac gtgaaagtga agtccatcaa tggcggcttc | 2040 |
| accagctttc tgcggcggaa gtggaagttt aagaaagagc ggaacaaggg gtacaagcac | 2100 |
| cacgccgagg acgccctgat cattgccaac gccgatttca tcttcaaaga gtggaagaaa | 2160 |
| ctggacaagg ccaaaaaagt gatggaaaac cagatgttcg aggaaaagca ggccgagagc | 2220 |
| atgcccgaga tcgaaaccga gcaggagtac aaagagatct tcatcacccc ccaccagatc | 2280 |
| aagcacatta aggacttcaa ggactacaag tacagccacc gggtggacaa gaagcctaat | 2340 |
| agagagctga ttaacgacac cctgtactcc acccggaagg acgacaaggg caacaccctg | 2400 |
| atcgtgaaca atctgaacgg cctgtacgac aaggacaatg acaagctgaa aaagctgatc | 2460 |
| aacaagagcc ccgaaaagct gctgatgtac caccacgacc cccagaccta ccagaaactg | 2520 |
| aagctgatta tggaacagta cggcgacgag aagaatcccc tgtacaagta ctacgaggaa | 2580 |
| accgggaact acctgaccaa gtactccaaa aaggacaacg gccccgtgat caagaagatt | 2640 |
| aagtattacg gcaacaaact gaacgcccat ctggacatca ccgacgacta ccccaacagc | 2700 |
| agaaacaagg tcgtgaagct gtccctgaag ccctacagat tcgacgtgta cctggacaat | 2760 |
| ggcgtgtaca agttcgtgac cgtgaagaat ctggatgtga tcaaaaaaga aaactactac | 2820 |
| gaagtgaata gcaagtgcta tgaggaagct aagaagctga agaagatcag caaccaggcc | 2880 |
| gagtttatcg cctccttcta caacaacgat ctgatcaaga tcaacggcga gctgtataga | 2940 |
| gtgatcggcg tgaacaacga cctgctgaac cggatcgaag tgaacatgat cgacatcacc | 3000 |
| taccgcgagt acctggaaaa catgaacgac aagaggcccc ccaggatcat taagacaatc | 3060 |
| gcctccaaga cccagagcat taagaagtac agcacagaca ttctgggcaa cctgtatgaa | 3120 |
| gtgaaatcta agaagcaccc tcagatcatc aaaaagggc | 3159 |

An exemplary codon optimized nucleic acid sequences encoding a Cas9 molecule
of S. aureus (SEQ ID NO: 9).

| | |
|---|---|
| atgaagcgca actacatcct cggactggac atcggcatta cctccgtggg atacggcatc | 60 |
| atcgattacg aaactaggga tgtgatcgac gctggagtca ggctgttcaa agaggcgaac | 120 |
| gtggagaaca acgaggggcg gcgctcaaag aggggggccc gccggctgaa gcgccgccgc | 180 |
| agacatagaa tccagcgcgt gaagaagctg ctgttcgact acaaccttct gaccgaccac | 240 |
| tccgaacttt ccggcatcaa cccatatgag gctagagtga agggattgtc ccaaaagctg | 300 |
| tccgaggaag agttctccgc cgcgttgctc cacctcgcca agcgcagggg agtgcacaat | 360 |
| gtgaacgaag tggaagaaga taccggaaac gagctgtcca ccaaggagca gatcagccgg | 420 |
| aactccaagg ccctggaaga gaaatacgtg gcggaactgc aactggagcg gctgaagaaa | 480 |
| gacggagaag tgcgcggctc gatcaaccgc ttcaagacct cggactacgt gaaggaggcc | 540 |
| aagcagctcc tgaaagtgca aaaggcctat caccaacttg accagtcctt tatcgatacc | 600 |
| tacatcgatc tgctcgagac tcggcggact tactacgagg gtccagggga gggctcccca | 660 |
| tttggttgga aggatattaa ggagtggtac gaaatgctga tgggacactg cacatacttc | 720 |
| cctgaggagc tgcggagcgt gaaatacgca tacaacgcag acctgtacaa cgcgctgaac | 780 |
| gacctgaaca atctcgtgat cacccgggac gagaacgaaa agctcgagta ttacgaaaag | 840 |
| ttccagatta ttgagaacgt gttcaaacag aagaagaagc cgacactgaa gcagattgcc | 900 |
| aaggaaatcc tcgtgaacga agaggacatc aagggctatc gagtgacctc aacgggaaag | 960 |
| ccggagttca ccaatctgaa ggtctaccac gacatcaaag acattaccgc ccggaaggag | 1020 |
| atcattgaga acgcggagct gttggaccag attgcgaaga ttctgaccat ctaccaatcc | 1080 |
| tccgaggata ttcaggaaga actcaccaac ctcaacagcg aactgaccca ggaggagata | 1140 |
| gagcaaatct ccaacctgaa gggctacacc ggaactcata acctgagcct gaaggccatc | 1200 |
| aacttgatcc tggacgagct gtggcacacc aacgataacc agatcgctat tttcaatcgg | 1260 |
| ctgaagctgg tccccaagaa agtggacctc tcacaacaaa aggagatccc tactaccctt | 1320 |
| gtggacgatt tcattctgtc ccccgtggtc aagagaagct tcatacagtc aatcaaagtg | 1380 |
| atcaatgcca ttatcaagaa atacggtctg cccaacgaca ttatcattga gctcgcccgc | 1440 |
| gagaagaact cgaaggacgc ccagaagatg attaacgaaa tgcagaagag gaaccgacag | 1500 |
| actaacgaac ggatcgaaga aatcatccgg accaccggga aggaaaacgc gaagtacctg | 1560 |
| atcgaaaaga tcaagctcca tgacatgcag gaaggaaagt gtctgtactc gctggaggcc | 1620 |
| attccgctgg aggacttgct gaacaaccct tttaactacg aagtggatca tatcattccg | 1680 |
| aggagcgtgt cattcgacaa ttccttcaac aacaaggtcc tcgtgaagca ggaggaaaac | 1740 |
| tcgaagaagg gaaaccgcac gccgttccag tacctgagca gcagcgactc caagatttcc | 1800 |
| tacgaaacct tcaagaagca catcctcaac ctggcaaagg ggaagggtcg catctccaag | 1860 |
| accaagaagg aatatctgct ggaagaaaga gacatcaaca gattctccgt gcaaaaggac | 1920 |
| ttcatcaacc gcaacctcgt ggatactaga tacgctactc ggggtctgat gaacctcctg | 1980 |
| agaagctact ttagagtgaa caatctggac gtgaaggtca agtcgattaa cggaggtttc | 2040 |
| acctccttcc tgcggcgcaa gtggaagttc aagaaggaac ggaacaaggg ctacaagcac | 2100 |
| cacgccgagg acgccctgat cattgccaac gccgacttca tcttcaaaga atggaagaaa | 2160 |
| cttgacaagg ctaagaaggt catggaaaac cagatgttcg aagaaaagca ggccgagtct | 2220 |
| atgcctgaaa tcgagactga acaggagtac aaggaaatct ttattacgcc acaccagatc | 2280 |
| aaacacatca aggatttcaa ggattacaag tactcacatc gcgtggacaa aaagccgaac | 2340 |
| agggaactga tcaacgacac cctctactcc acccggaagg atgacaaagg gaataccctc | 2400 |
| atcgtcaaca accttaacgg cctgtacgac aaggacaacg ataagctgaa gaagctcatt | 2460 |
| aacaagtcgc ccgaaaagtt gctgatgtac caccacgacc ctcagactta ccagaagctc | 2520 |
| aagctgatca tggagcagta tggggacgag aaaaacccgt tgtacaagta ctacgaagaa | 2580 |
| actgggaatt atctgactaa gtactccaag aaagataacg gccccgtgat taagaagatt | 2640 |
| aagtactacg gcaacaagct gaacgcccat ctggacatca ccgatgacta ccctaattcc | 2700 |
| cgcaacaagg tcgtcaagct gagcctcaag ccctaccggt ttgatgtgta ccttgacaat | 2760 |
| ggagtgtaca agttcgtgac tgtgaagaac cttgacgtga tcaagaagga gaactactac | 2820 |
| gaagtcaact ccaagtgcta cgaggaagca aagaagttga agaagatctc gaaccaggcc | 2880 |
| gagttcattg cctccttcta taacaacgac ctgattaaga tcaacggcga actgtaccgc | 2940 |
| gtcattggcg tgaacaacga tctcctgaac cgcatcgaag tgaacatgat cgacatcact | 3000 |
| taccgggaat acctggagaa tatgaacgac aagcgcccgc cccggatcat taagactatc | 3060 |
| gcctcaaaga cccagtcgat caagaagtac agcaccgaca tcctgggcaa cctgtacgag | 3120 |
| gtcaaatcga agaagcaccc ccagatcatc aagaaggga | 3159 |

1. A system for selecting a version of an RNA guided nuclease, comprising:
a library of polynucleotides, wherein different polynucleotides in the library encode different versions of the RNA guided nuclease; and
a plurality of bacteriophage comprising a phagemid that encodes a first selection agent and includes a DNA target site,
wherein the DNA target site comprises (i) a canonical or non-canonical protospacer adjacent motif (PAM) and (ii) a target site for a guide RNA,
wherein the library of polynucleotides or the plurality of bacteriophage further comprises the guide RNA, and
such that, upon incubation of (1) host cells transformed with the library with (2) the plurality of bacteriophage:
(i) the plurality of bacteriophage infect the transformed host cells, wherein expression of the first selection agent confers a survival disadvantage in infected host cells and binding at the DNA target site decreases expression of the first selection agent, and host cells that were transformed with a plasmid that encodes a version of the RNA guided nuclease that binds the DNA target site survive while host cells that were transformed with a plasmid that encodes a version of the RNA guided nuclease that does not bind the DNA target site do not survive; or
(ii) the plurality of bacteriophage infect the transformed host cells, wherein expression of the first selection agent confers a survival advantage in infected host cells and binding at the DNA target site decreases expression of the first selection agent, and host cells that were transformed with a plasmid that encodes a version of the RNA guided nuclease that does not bind the DNA target site survive while host cells that were transformed with a plasmid that encodes a version of the RNA guided nuclease that binds the DNA target site do not survive; or
(iii) the plurality of bacteriophage infect the transformed host cells, wherein expression of the first selection agent confers a survival advantage in infected host cells and binding at the DNA target site increases expression of the first selection agent, and host cells that were transformed with a plasmid that encodes a version of the RNA guided nuclease that binds the DNA target site survive while host cells that were transformed with a plasmid that encodes a version of the RNA guided nuclease that does not bind the DNA target site do not survive; or
(iv) the plurality of bacteriophage infect the transformed host cells, wherein expression of the first selection agent confers a survival disadvantage in infected host cells and binding at the DNA target site increases expression of the first selection agent, and host cells that were transformed with a plasmid that encodes a version of the RNA guided nuclease that does not bind the DNA target site survive while host cells that were transformed with a plasmid that encodes a version of the RNA guided nuclease that binds the DNA target site do not survive.

2-3. (canceled)

4. The system of claim 1 wherein the target site comprises a sequence that is complementary to the gRNA sequence.

5. A method of selecting a version of a polypeptide or polynucleotide of interest based on whether it binds a DNA target site, the method comprising steps of:
(a) introducing a library of polynucleotides into host cells, wherein different polynucleotides in the library encode different versions of the polypeptide of interest or serve as templates for different versions of the polynucleotide of interest, so that each transformed host cell includes a polynucleotide that encodes a version of the polypeptide of interest or serves as a template for a version of the polynucleotide of interest;
(b) incubating transformed host cells from step (a) together with a plurality of bacteriophage comprising a phagemid that encodes a first selection agent and includes a DNA target site, under culture conditions such that:
(i) the plurality of bacteriophage infect the transformed host cells, wherein expression of the first selection agent confers a survival disadvantage in infected host cells and binding at the DNA target site decreases expression of the first selection agent, and host cells that were transformed with a plasmid that encodes a version of the polypeptide or polynucleotide of interest that binds the DNA target site survive while host cells that were transformed with a plasmid that encodes a version of the polypeptide or polynucleotide of interest that does not bind the DNA target site do not survive; or
(ii) the plurality of bacteriophage infect the transformed host cells, wherein expression of the first selection agent confers a survival advantage in infected host cells and binding at the DNA target site decreases expression of the first selection agent, and host cells that were transformed with a plasmid that encodes a version of the polypeptide or polynucleotide of interest that does not bind the DNA target site survive while host cells that were transformed with a plasmid that encodes a version of the polypeptide or polynucleotide of interest that binds the DNA target site do not survive; or
(iii) the plurality of bacteriophage infect the transformed host cells, wherein expression of the first selection agent confers a survival advantage in infected host cells and binding at the DNA target site increases expression of the first selection agent, and host cells that were transformed with a plasmid that encodes a version of the polypeptide or polynucleotide of interest that binds the DNA target site survive while host cells that were transformed with a plasmid that encodes a version of the polypeptide or polynucleotide of interest that does not bind the DNA target site do not survive; or
(iv) the plurality of bacteriophage infect the transformed host cells, wherein expression of the first selection agent confers a survival disadvantage in infected host cells and binding at the DNA target site increases expression of the first selection agent, and host cells that were transformed with a plasmid that encodes a version of the polypeptide or polynucleotide of interest that does not bind the DNA target site survive while host cells that were transformed with a plasmid that encodes a version of the polypeptide or polynucleotide of interest that binds the DNA target site do not survive; and
(c) selecting for host cells that survive step (b).

6-9. (canceled)

10. The method of claim 5, wherein the polynucleotide further comprises a regulatory element operably linked to the polypeptide encoding the polypeptide or to the polynucleotide of interest.

11-12. (canceled)

13. The method of claim 5, wherein binding at the DNA target site decreases expression of the first selection agent by cleaving the phagemid at or near the DNA target site.

14-15. (canceled)

16. The method of claim 5, wherein the phagemid includes a regulatory element that drives expression of the first selection agent.

17. The method of claim 16, wherein the DNA target site is located within the regulatory element.

18. The method of claim 17, wherein binding at the DNA target site decreases expression of the first selection agent by repressing expression of the first selection agent.

19-27. (canceled)

28. The method of claim 5, wherein the culture conditions include culturing transformed host cells from step (b) together with the plurality of bacteriophage in a competitive culture.

29-31. (canceled)

32. The method of claim 5, wherein the library of polynucleotides is a library of plasmids.

33. (canceled)

34. The method of claim 5, wherein the first selection agent is a toxin.

35-39. (canceled)

40. The method of claim 5, wherein the first selection agent is encoded by an antibiotic resistance gene and the culture conditions include exposure to an antibiotic to which the antibiotic resistance gene provides resistance.

41-42. (canceled)

43. The method of claim 5, wherein the host cells are bacterial cells.

44-47. (canceled)

48. The method of claim 5, wherein the polypeptide or polynucleotide of interest is a nuclease.

49. The method of claim 48, wherein the nuclease is a CRISPR-associated (Cas) nuclease and polynucleotides in the library further comprise a DNA sequence that encodes a gRNA that localizes the Cas nuclease to the DNA target site.

50. The method of claim 49, wherein the Cas nuclease is a Cas9 nuclease.

51. The method of claim 49, wherein the Cas nuclease is a Cpf1 nuclease.

52-67. (canceled)

68. The method of claim 13, wherein the cleaving comprises cleaving one strand of the phagemid.

69. The method of claim 13, wherein the cleaving comprises cleaving both strands of the phagemid.

70. (canceled)

71. A library comprising a plurality of bacteriophage comprising a phagemid that encodes a first selection agent and comprises a DNA target site for a preselected polypeptide or polynucleotide of interest.

72-78. (canceled)
